(19) United States
(12) Patent Application Publication
(10) Pub. No.: US 2006/0121475 A1
(43) Pub. Date: Jun. 8, 2006

(54) Protein kinases

(75) Inventors: Andrew Robert Davids, London (GB); Christopher Benjamin Phelps, London (GB); Richard Joseph Fagan, London (GB); Christine Power, Thoiry (FR); Melanie Yorke, Confignon (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Ares Trading S.A., Vaumarcus (CH)

(21) Appl. No.: 10/526,722

(22) PCT Filed: Sep. 5, 2003
(86) PCT No.: PCT/GB03/03862

§ 371 Date: Sep. 2, 2005

(30) Foreign Application Priority Data
Sep. 6, 2002 (GB) 0220770.2

(51) Int. Cl. C12Q 1/68; C07H 21/04; C12P 21/06; C12N 9/12

(52) U.S. Cl. 435/6; 435/69.1; 435/194; 435/320.1; 435/325; 536/23.2

(57) ABSTRACT

The present invention relates to novel proteins, termed INSP081, INSP082, and INSP091, herein identified as members of the Germinal Center Kinase (GCK) subfamily of the STE20 family of protein kinases, preferably as NCK-interacting kinases (NIKs) and more preferably as NIK-like embryo specific kinases (NESKs), and to the use of these and nucleic acid sequences from the encoding genes in the diagnosis, prevention, and treatment of disease.

This invention relates to novel proteins, termed INSP081, INSP082 and INSP091, herein identified as members of the Germinal Center Kinase (GCK) subfamily of the STE20 family of protein kinases, preferably as NCK-interacting kinases (NIKs) and more preferably as NIK-like embryo specific kinases (NESKs), and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

Introduction

Protein Kinases

Protein kinases catalyse the transfer of a phosphate from ATP to an amino acid residue of protein targets. They are involved in all aspects of signal transduction in eukaryotic cells, from primary transmembrane signalling to control of transcription and the cell cycle.

Protein kinases are classified as Tyrosine kinases and/or Serine/Threonine kinases by the target protein residue that receives the phosphoryl group: either tyrosine or serine or threonine (Hanks, S. K. And Hunter, T. (1995) FASEB 9:576-596).

Tyrosine kinases can be grouped into two categories. The first category is tyrosine kinases that have a transmembrane segment, which are usually receptors for extracellular ligands (receptor tyrosine kinases), e.g. Insulin receptor that is involved in various forms of insulin resistance as well as some forms of diabetes mellitus.

Proteins of the second category are nonreceptor tyrosine kinases, which are only intracellular; one example is the proto-oncogene tyrosine-protein kinase ABL. Alterations of the ABL1 gene by chromosomal rearrangement or viral transduction lead to chronic myeloid leukaemia.

Serine/Threonine kinases phosphorylate a number of protein substrates resulting in the activation or inactivation of the protein. Examples of this include enzymes, transcription factors, cytoskeletal proteins, receptors and ion channels. These proteins play a key role in all cellular processes, such as apoptosis, cell cycle and transcription (for review see Kolch W., Biochem J. Oct, 15, 2000;351(Pt 2):289-305, Davie J R, Spencer V A., Prog Nucleic Acid Res Mol Biol. 2000;65:299-340, Ham J, et al, Biochem Pharmacol. 2000;60(8):1015-21, Reed J C, Bischoff J R., Cell. 2000;102(5):545-8, Saxena M, Mustelin T., Semin Immunol. 2000;12(4):387-96). Serine/Threonine-protein kinase CHK1 for example, is a nuclear protein involved in cell cycle arrest when DNA damage has occured or when unligated DNA is present, by binding to and phosphorylating CDC25 proteins (Sanchez Y. et al., (1997) Science 277:1497-1501).

Kinases, both serine/threonine and tyrosine have been directly implicated in a variety of diseases, encompassing all therapeutic areas such as oncology (Mimori K, et al, Ann Surg Oncol. 2000;7(9):692-5, Erickson L A, et al, Mod Pathol. 2000;13(9): 1014-9, Hennige A M, et al., Mol Cell Endocrinol. 2000;167(1-2):69-76, Harrington E O, et al., Am J Physiol Lung Cell Mol Physiol. 2000;279(4):L733-42, Amin H M, et al., Br J Haematol. 2000;110(3):552-62, Tang X, et al., J Natl Cancer Inst. 2000;92(18):1511-1516, Drevs J, et al., Cancer Res. 2000;60(17):4819-24), metabolism (Coghlan M. P., et al, Chem Biol. 30 2000;7(10):793-803, Coghlan M P, et al., Chem Biol. 2000 7(10):793-803, Waeber G, et al., Nat Genet. 2000;24(3):291-5), central nervous system (CNS) (Tan J., et al., J Neurosci. 2000 20(20):7587-94, Leclerc S, et al., J Biol Chem. 2000 275: 30144-30152), cardiovascular (Mounsey J P, et al,. Hum Mol Genet. 2000;9(15):2313-20, Sanz-Gonzalez S M, et al., Front Biosci. 2000;5:D619-28, Petkova S B, et al., Front Biosci. 2000;5:D452-60), inflammation (Lee S. J., et al, J Immunol. 2000;165(8):4658-4666, Fiebich B. L., et al, J Neurochem. 2000;75(5):2020-2028, Barchowsky A, et al, Cytokine 2000;12(10):1469-1479, Cuzzocrea S, et al, Lab Invest. 2000;80(9):1439-53), and infection (Warny M, et al., J Clin Invest. 2000;105(8):1147-56, Read T D, et al., Nucleic Acids Res. 2000;28(6):1397-406).

STE-20 Kinase Family

The STE20 kinase family of protein kinases is involved in the regulation of the c-Jun N-terminal kinase pathway. STE20 was initially discovered as S. cerevisiae MAP4K. A number of mammalian subfamilies have since been discovered. One of these is the Germinal Center Kinase subfamily of STE20 kinases.

Germinal Center Kinase Subfamily

The GCK subfamily of STE20 kinases is itself divided up into 2 subfamilies with respect to JNK activation (Kyriakis. J. M. (1999) Signaling by the germinal center kinase family of protein kinases. J. Biol. Chem, 274, 5259-5262).

Group 1 GCKs include GCK, GCK related kinase (GCKR), hematopoietic progenitor kinase 1 (HPK1), GCK-like kinase (GLK), HPK/GCK-like kinase and NCK-interacting kinase (NIK).

The group 1 GCK family members mentioned above have been shown to activate the JNK pathway (Diener, K. et al. (1997) Activation of the c-Jun N-terminal kinase pathway by a novel protein kinase related to human germinal center kinase. Proc. Natl. Acad. Sci. USA., 94, 9687-9692; Hu, M. C. et al. (1996) Human HPK1 , a novel hematopoietic progenitor kinase that activates the JNK/SAPK kinase cascade. Genes Dev. 10, 2251-2264; Pombo, C. M. et al. (1995) Activation of the SAPK pathway by the human STE-20 homologue germinal centre kinase. Nature, 377, 750-754; Su et al. (1997) NIK is a new STE20-related kinase that binds NCK and MEKK1 and activates the SAPK/JNK cascade via a conserved regulatory domain. EMBO J. 16, 1279-1290).

All members of the group 1 GCK family are known to activate selectively the JNK pathway but not the ERK1 or p38-MAPK pathways when expressed in cultured cells (Pombo, C. M. et al. (1995) Activation of the SAPK pathway by the human STE20 homologue germinal centre kinase. Nature, 377, 750-754).

Members of the group 1 GCK family have an N-terminal kinase domain which resembles the catalytic domain found in STE20, a variable intermediate region containing at least two proline-rich Src homology 3 (SH3) domains and a C-terminal regulatory region. The C-terminal domain further comprises a domain distantly related to part of the murine citron protein (citron homology (CNH) domain). This C-terminal domain may function to couple these kinases to downstream MAP3Ks (Su et al. (1997) NIK is a new STE20-related kinase that binds NCK and MEKK1 and activates the SAPK/JNK cascade via a conserved regulatory domain. EMBO J. 16, 1279-1290).

NIK has been shown to interact with NCK, a Src homology 2-Src homology 3 (SH2-SH3) domain containing protein (Su et al. (1997) NIK is a new STE20-related kinase that binds NCK and MEKK1 and activates the SAPK/JNK cascade via a conserved regulatory domain. EMBO J. 16, 1279-1290). It is proposed to link protein tyrosine kinase signals to JNK activation and may play a role in cytoskeletal regulation (Xue et al. Mesodermal patterning defects in mice lacking the STE20 NCK interacting kinase (NIK), Development (2000), 128, 1559-1572).

NIK has been found to be essential for development in mammalian cells. Nik^{-/-} mouse embryos have been show to die postgastrulation. Patterning experiments in mice have suggested that NIK plays a critical and specific role in regulating the migration of cells that arise from the region of the primitive streak just posterior to the node (Xue et al. (2000) Mesodermal patterning defects in mice lacking the STE20 NCK interacting kinase (NIK), Development, 128, 1559-1572). These experiments also led to the suggestion that NIK may regulate the mesodermal migration that contributes to the elongation of the body axis. Xue et al. have further speculated that a NCK/NIK complex may be required for segmentation of presomitic mesoderm into somites.

NESK (NIK-like embryo specific kinase) is a further member of the GCK family subgroup (Nakano et al. NESK, a member of the Germinal center kinase family that activates the c-Jun N-terminal kinase pathway and is expressed during the late stages of embryogenesis. JBC, (2000) 275, 27, 20533-20539). NESK is expressed exclusively during the late stages of embryogenesis. NESK has been shown to activate the JNK pathway when overexpressed in HEK293 (Nakano et al. JBC, (2000) 275, 27, 20533-20539). Although maximal activation of the JNK pathway requires the C-terminal domain of GCK, the kinase domain alone of NESK was able to activate the JNK pathway (Nakano et al. JBC, (2000) 275, 27, 20533-20539). Nakano et al. have suggested that NESK may play a role in coupling TNF receptor-associated factor 2 (TRAF2), as well as TNF-α, to JNK activation (Nakano et al. (2000) NESK, a member of the germinal center kinase family that activates the c-Jun N-terminal kinase pathway and is expressed during the late stages of embryogenesis. JBC, 275(27), 20533-20539). It has been proposed that NESK functions as an intracellular signalling molecule in developmental processes occurring late in the embryogenetic process (Nakano, K. et al. (2000) NESK, a member of the germinal center kinase family that activates the c-Jun N-terminal kinase pathway and is expressed during the late stages of embryogenesis. JBC, 275 (27), 20533-20539).

As NIK kinases play such a key role in cellular signalling processes, it is not surprising that dysregulation of these kinases impinges on a variety of disorders. In particular, dysregulation of NESK kinases is likely to result in a disorder of the late stage of embryogenetic development. Identification of NIK and NESK kinases is therefore of extreme importance in increasing understanding of the underlying pathways that lead to the disorders mentioned above and in developing more effective gene or drug therapies to treat these disorders.

### THE INVENTION

The invention is based on the discovery that the human INSP081, INSP082 and INSP091 polypeptides are members of the Germinal Center Kinase (GCK) subfamily of the STE20 family of protein kinases, preferably NIK-like kinases and more preferably NIK-like embryo specific kinases (NESK).

In one embodiment of the first aspect of the invention, there is provided a polypeptide which:
(i) comprises the amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40 and/or SEQ ID NO:42;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

Preferably, the polypeptide according to this first embodiment of the first aspect of the invention:
(i) comprises the amino acid sequence as recited in SEQ ID NO:42;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

According to a second embodiment of this first aspect of the invention, there is provided a polypeptide which:
(i) consists of the amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40 and/or SEQ ID NO:42;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or having an antigenic determinant in common with the polypeptides of (i); or
(iii) is a functional equivalent of (i) or (ii).

The polypeptide having the sequence recited in SEQ ID NO:2 is referred to hereafter as “INSP081 exon 1 polypeptide”. The polypeptide having the sequence recited in SEQ ID NO:4 is referred to hereafter as “INSP081 exon 2 polypeptide”. The polypeptide having the sequence recited in SEQ ID NO:6 is referred to hereafter as “INSP081 exon 3 polypeptide”. The polypeptide having the sequence recited in SEQ ID NO:8 is referred to hereafter as “INSP081 exon 4 polypeptide”. The polypeptide having the sequence recited in SEQ ID NO:10 is referred to hereafter as “INSP081 exon 5 polypeptide”. The polypeptide having the sequence recited in SEQ ID NO:12 is referred to hereafter as “INSP081 exon 6 polypeptide”. The polypeptide having the sequence recited in SEQ ID NO: 14 is referred to hereafter as “INSP081 exon 7 polypeptide”. The polypeptide having the sequence recited in SEQ ID NO:16 is referred to hereafter as “INSP081 exon 8 polypeptide”. The polypeptide having the sequence recited in SEQ ID NO: 18 is referred to hereafter as “INSP081 exon 9 polypeptide”. The polypeptide having the sequence recited in SEQ ID NO:20 is referred to hereafter as “INSP081 exon 10 polypeptide”. The polypeptide having the sequence recited in SEQ ID NO:22 is referred to hereafter as “INSP081 exon 11 polypeptide”. The polypeptide having the sequence recited in SEQ ID NO:24 is referred to hereafter as “INSP081 exon 12 polypeptide”. The polypeptide having the sequence recited in SEQ ID NO:26 is referred to hereafter as “INSP081 exon 13 polypeptide”. The polypeptide having the sequence recited in SEQ ID NO:28 is referred to hereafter as “INSP081 exon 14 polypeptide”. The polypeptide having the sequence recited in SEQ ID NO:30 is referred to hereafter as “INSP081 exon 15 polypeptide”. The polypeptide having the sequence recited in SEQ ID NO:32 is referred to hereafter as “INSP081 exon 16 polypeptide”. The polypeptide having the sequence recited in SEQ ID NO:34 is referred to hereafter as “INSP081 exon 17 polypeptide”. The polypeptide having the sequence recited in SEQ ID NO:36 is referred to hereafter as “INSP081 exon 18 polypeptide”. The polypeptide having the sequence recited in SEQ ID NO:38 is referred to hereafter as “INSP081 exon 19 polypeptide”. The polypeptide having the sequence recited in SEQ ID NO:40 is referred to hereafter as “INSP081 exon 20 polypeptide”. The polypeptide having the sequence recited in SEQ ID NO:42 is referred to hereafter as the “INSP081 polypeptide”.

The term “INSP081 polypeptides” as used herein includes polypeptides comprising the INSP081 exon 1 polypeptide, the INSP081 exon 2 polypeptide, the INSP081 exon 3 polypeptide, the INSP081 exon 4 polypeptide, the INSP081 exon 5 polypeptide, the INSP081 exon 6 polypeptide, the INSP081 exon 7 polypeptide, the INSP081 exon 8 polypeptide, the INSP081 exon 9 polypeptide, the INSP081 exon 10 polypeptide, the INSP081 exon 11 polypeptide, the INSP081 exon 12 polypeptide, the INSP081 exon 13 polypeptide, the INSP081 exon 14 polypeptide, the INSP081 exon 15 polypeptide, the INSP081 exon 16 polypeptide, the INSP081 exon 17 polypeptide, the INSP081 exon 18 polypeptide, the INSP081 exon 19 polypeptide, the INSP081 exon 20 polypeptide and the INSP081 polypeptide.

In a third embodiment of the first aspect of the invention, there is provided a polypeptide which:
(i) comprises the amino acid sequence as recited in SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98 and/or SEQ ID NO:100;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

Preferably, the polypeptide according to this third embodiment of the first aspect of the invention:
(i) comprises the amino acid sequence as recited in SEQ ID NO:100;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

According to a fourth embodiment of this first aspect of the invention, there is provided a polypeptide which:
(i) consists of the amino acid sequence as recited in SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98 and/or SEQ ID NO:100;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

The polypeptide having the sequence recited in SEQ ID NO:44 is referred to hereafter as “INSP082 exon 1 polypeptide”. The polypeptide having the sequence recited in SEQ ID NO:46 is referred to hereafter as “INSP082 exon 2 polypeptide”. The polypeptide having the sequence recited in SEQ ID NO:48 is referred to hereafter as “INSP082 exon 3 polypeptide”. The polypeptide having the sequence recited in SEQ ID NO:50 is referred to hereafter as “INSP082 exon 4 polypeptide”. The polypeptide having the sequence recited in SEQ ID NO:52 is referred to hereafter as “INSP082 exon 5 polypeptide”. The polypeptide having the sequence recited in SEQ ID NO:54 is referred to hereafter as “INSP082 exon 6 polypeptide”. The polypeptide having the sequence recited in SEQ ID NO:56 is referred to hereafter as “INSP082 exon 7 polypeptide”. The polypeptide having the sequence recited in SEQ ID NO:58 is referred to hereafter as “INSP082 exon 8 polypeptide”. The polypeptide having the sequence recited in SEQ ID NO:60 is referred to hereafter as “INSP082 exon 9 polypeptide”. The polypeptide having the sequence recited in SEQ ID NO:62 is referred to hereafter as “INSP082 exon 10 polypeptide”. The polypeptide having the sequence recited in SEQ ID NO:64 is referred to hereafter as “INSP082 exon 11 polypeptide”. The polypeptide having the sequence recited in SEQ ID NO:66 is referred to hereafter as “INSP082 exon 12 polypeptide”. The polypeptide having the sequence recited in SEQ ID NO:68 is referred to hereafter as “INSP082 exon 13 polypeptide”. The polypeptide having the sequence recited in SEQ ID NO:70 is referred to hereafter as “INSP082 exon 14 polypeptide”. The polypeptide having the sequence recited in SEQ ID NO:72 is referred to hereafter as “INSP082 exon 15 polypeptide”. The polypeptide having the sequence recited in SEQ ID NO:74 is referred to hereafter as “INSP082 exon 16 polypeptide”. The polypeptide having the sequence recited in SEQ ID NO:76 is referred to hereafter as “INSP082 exon 17 polypeptide”. The polypeptide having the sequence recited in SEQ ID NO:78 is referred to hereafter as “INSP082 exon 18 polypeptide”. The polypeptide having the sequence recited in SEQ ID NO:80 is referred to hereafter as “INSP082 exon 19 polypeptide”. The polypeptide having the sequence recited in SEQ ID NO:82 is referred to hereafter as “INSP082 exon 20 polypeptide”. The polypeptide having the sequence recited in SEQ ID NO:84 is referred to hereafter as “INSP082 exon 21 polypeptide”. The polypeptide having the sequence recited in SEQ ID NO:86 is referred to hereafter as “INSP082 exon 22 polypeptide”. The polypeptide having the sequence recited in SEQ ID NO:88 is referred to hereafter as “INSP082 exon 23 polypeptide”. The polypeptide having the sequence recited in SEQ ID NO:90 is referred to hereafter as “INSP082 exon 24 polypeptide”. The polypeptide having the sequence recited in SEQ ID NO:92 is referred to hereafter as “INSP082 exon 25 polypeptide”. The polypeptide having the sequence recited in SEQ ID NO:94 is referred to hereafter as “INSP082 exon 26 polypeptide”. The polypeptide having the sequence recited in SEQ ID NO:96 is referred to hereafter as “INSP082 exon 27 polypeptide”. The polypeptide having the sequence recited in SEQ ID NO:98 is referred to hereafter as “INSP082 exon 28 polypeptide”. The polypeptide having the sequence recited in SEQ ID NO: 100 is referred to hereafter as the “INSP082 polypeptide”.

The term “INSP082 polypeptides” as used herein includes polypeptides comprising the INSP082 exon 1 polypeptide, the INSP082 exon 2 polypeptide, the INSP082 exon 3 polypeptide, the INSP082 exon 4 polypeptide, the INSP082 exon 5 polypeptide, the INSP082 exon 6 polypeptide, the INSP082 exon 7 polypeptide, the INSP082 exon 8 polypeptide, the INSP082 exon 9 polypeptide, the INSP082 exon 10 polypeptide, the INSP082 exon 11 polypeptide, the INSP082 exon 12 polypeptide, the INSP082 exon 13 polypeptide, the INSP082 exon 14 polypeptide, the INSP082 exon 15 polypeptide, the INSP082 exon 16 polypeptide, the INSP082 exon 17 polypeptide, the INSP082 exon 18 polypeptide, the INSP082 exon 19 polypeptide, the INSP082 exon 20 polypeptide, the INSP082 exon 21 polypeptide, the INSP082 exon 22 polypeptide, the INSP082 exon 23 polypeptide, the INSP082 exon 24 polypeptide, the INSP082 exon 25 polypeptide, the INSP082 exon 26 polypeptide, the INSP082 exon 27 polypeptide, the INSP082 exon 28 polypeptide and the INSP082 polypeptide.

In a fifth embodiment of the first aspect of the invention, there is provided a polypeptide which:
(i) comprises the amino acid sequence as recited in SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 and/or SEQ ID NO:158;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

Preferably, the polypeptide according to this fifth embodiment of the first aspect of the invention:
(i) comprises the amino acid sequence as recited in SEQ ID NO:158;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

According to a sixth embodiment of this first aspect of the invention, there is provided a polypeptide which:
(i) consists of the amino acid sequence as recited in SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 and/or SEQ ID NO:158;
(ii) is a fragment thereof which is a member of the Germinal Center Kinase (GCK) subfamily of the STE20 family of protein kinases, preferably a NIK-like kinase and more preferably a NIK-like embryo specific kinase (NESK), or has an antigenic determinant in common with the polypeptides of (i); or
(iii) is a functional equivalent of (i) or (ii).

The polypeptide having the sequence recited in SEQ ID NO:102 is referred to hereafter as “INSP091 exon 1 polypeptide”. The polypeptide having the sequence recited in SEQ ID NO:104 is referred to hereafter as “INSP091 exon 2 polypeptide”. The polypeptide having the sequence recited in SEQ ID NO:106 is referred to hereafter as “INSP091 exon 3 polypeptide”. The polypeptide having the sequence recited in SEQ ID NO:108 is referred to hereafter as “INSP091 exon 4 polypeptide”. The polypeptide having the sequence recited in SEQ ID NO: I10 is referred to hereafter as “INSP091 exon 5 polypeptide”. The polypeptide having the sequence recited in SEQ ID NO:112 is referred to hereafter as “INSP091 exon 6 polypeptide”. The polypeptide having the sequence recited in SEQ ID NO:114 is referred to hereafter as “INSP091 exon 7 polypeptide”. The polypeptide having the sequence recited in SEQ ID NO:116 is referred to hereafter as “INSP091 exon 8 polypeptide”. The polypeptide having the sequence recited in SEQ ID NO:118 is referred to hereafter as “INSP091 exon 9 polypeptide”. The polypeptide having the sequence recited in SEQ ID NO:120 is referred to hereafter as “INSP091 exon 10 polypeptide”. The polypeptide having the sequence recited in SEQ ID NO:122 is referred to hereafter as “INSP091 exon 11 polypeptide”. The polypeptide having the sequence recited in SEQ ID NO:124 is referred to hereafter as “INSP091 exon 12 polypeptide”. The polypeptide having the sequence recited in SEQ ID NO:126 is referred to hereafter as “INSP091 exon 13 polypeptide”. The polypeptide having the sequence recited in SEQ ID NO:128 is referred to hereafter as “INSP091 exon 14 polypeptide”. The polypeptide having the sequence recited in SEQ ID NO:130 is referred to hereafter as “INSP091 exon 15 polypeptide”. The polypeptide having the sequence recited in SEQ ID NO:132 is referred to hereafter as “INSP091 exon 16 polypeptide”. The polypeptide having the sequence recited in SEQ ID NO:134 is referred to hereafter as “INSP091 exon 17 polypeptide”. The polypeptide having the sequence recited in SEQ ID NO:136 is referred to hereafter as “INSP091 exon 18 polypeptide”. The polypeptide having the sequence recited in SEQ ID NO:138 is referred to hereafter as “INSP091 exon 19 polypeptide”. The polypeptide having the sequence recited in SEQ ID NO:140 is referred to hereafter as “INSP091 exon 20 polypeptide”. The polypeptide having the sequence recited in SEQ ID NO:142 is referred to hereafter as “INSP091 exon 21 polypeptide”. The polypeptide having the sequence recited in SEQ ID NO:144 is referred to hereafter as “INSP091 exon 22 polypeptide”. The polypeptide having the sequence recited in SEQ ID NO:146 is referred to hereafter as “INSP091 exon 23 polypeptide”. The polypeptide having the sequence recited in SEQ ID NO:148 is referred to hereafter as “INSP091 exon 24 polypeptide”. The polypeptide having the sequence recited in SEQ ID NO:150 is referred to hereafter as “INSP091 exon 25 polypeptide”. The polypeptide having the sequence recited in SEQ ID NO:152 is referred to hereafter as “INSP091 exon 26 polypeptide”. The polypeptide having the sequence recited in SEQ ID NO:1 54 is referred to hereafter as “INSP091 exon 27 polypeptide”. The polypeptide having the sequence recited in SEQ ID NO:156 is referred to hereafter as “INSP091 exon 28 polypeptide”. The polypeptide having the sequence recited in SEQ ID NO:158 is referred to hereafter as the “INSP091 polypeptide”. The term “INSP091 polypeptides” as used herein includes polypeptides comprising the INSP091 exon 1 polypeptide, the INSP091 exon 2 polypeptide, the INSP091 exon 3 polypeptide, the INSP091 exon 4 polypeptide, the INSP091 exon 5 polypeptide, the INSP091 exon 6 polypeptide, the INSP091 exon 7 polypeptide, the INSP091 exon 8 polypeptide, the INSP091 exon 9 polypeptide, the INSP091 exon 10 polypeptide, the INSP091 exon 11 polypeptide, the INSP091 exon 12 polypeptide, the INSP091 exon 13 polypeptide, the INSP091 exon 14 polypeptide, the INSP091 exon 15 polypeptide, the INSP091 exon 16 polypeptide, the INSP091 exon 17 polypeptide, the INSP091 exon 18 polypeptide, the INSP091 exon 19 polypeptide, the INSP091 exon 20 polypeptide, the INSP091 exon 21 polypeptide, the INSP091 exon 22 polypeptide, the INSP091 exon 23 polypeptide, the INSP091 exon 24 polypeptide, the INSP091 exon 25 polypeptide, the INSP091 exon 26 polypeptide, the INSP091 exon 27 polypeptide, the INSP091 exon 28 polypeptide and the INSP091 polypeptide.

The INSP081 polypeptide is a splice variant of the INSP082 and INSP091 polypeptides. Whilst INSP081 contains a STOP codon over the exon 20/exon 21 splice junction, INSP082 does not. Thus, INSP081 contains 20 exons and is 1132 amino acids long whereas INSP082 contains 28 exons is 1500 amino acids long. The presence of the STOP codon in intron 20 truncates INSP081 such that it does not contain the putative C-terminal regulatory domain. INSP082 on the other hand is predicted to contain this C-terminal regulatory (CNH) domain. INSP091 is also predicted to contain the putative C-terminal regulatory domain, and is identical to the INSP081 and INSP082 polypeptides from its N-terminus until exon 15. After exon 15 the INSP091 polypeptide is not identical to the INSP081 and INSP082 polypeptides, although it has a similar number of exons and has a number of exons in common with the INSP081 and INSP082 polypeptides. The INSP091 polypeptide contains 28 exons and is 1589 amino acids long.

INSP081, INSP082 and INSP091 all map to the X chromosome at ChrX:101702493-101913965. INSP081, INSP082 and INSP091 each map to the same genomic locus as the human sequence ZC4 DNA disclosed in WO 99/53036. ZC4 DNA has been annotated as a STE20-related kinase. It has a similar number of exons to INSP081, INSP082 and INSP091 polypeptides and has a number of exons in common with the predicted proteins. However, the total complement of exons used by INSP081, INSP082 and INSP091 is different from the total complement of exons used by ZC4 DNA. The sequence of ZC4 DNA is specifically excluded from the scope of this invention.

In a second aspect, the invention provides a purified nucleic acid molecule which encodes a polypeptide of the first aspect of the invention.

In a first embodiment of the second aspect of the invention, the purified nucleic acid molecule comprises the nucleic acid sequence as recited in SEQ ID NO: I (encoding the INSP081 exon 1 polypeptide), SEQ ID NO:3 (encoding the INSP081 exon 2 polypeptide), SEQ ID NO:5 (encoding the INSP081 exon 3 polypeptide), SEQ ID NO:7 (encoding the INSP081 exon 4 polypeptide), SEQ ID NO:9 (encoding the INSP081 exon 5 polypeptide), SEQ ID NO:1 (encoding the INSP081 exon 6 polypeptide), SEQ ID NO:13 (encoding the INSP081 exon 7 polypeptide), SEQ ID NO:15 (encoding the INSP081 exon 8 polypeptide), SEQ ID NO:17 (encoding the INSP081 exon 9 polypeptide), SEQ ID NO:19 (encoding the INSP081 exon 10 polypeptide), SEQ ID NO:21 (encoding the INSP081 exon 11 polypeptide), SEQ ID NO:23 (encoding the INSP081 exon 12 polypeptide), SEQ ID NO:25 (encoding the INSP081 exon 13 polypeptide), SEQ ID NO:27 (encoding the INSP081 exon 14 polypeptide), SEQ ID NO:29 (encoding the INSP081 exon 15 polypeptide), SEQ ID NO:31 (encoding the INSP081 exon 16 polypeptide), SEQ ID NO:33 (encoding the INSP081 exon 17 polypeptide), SEQ ID NO:35 (encoding the INSP081 exon 18 polypeptide), SEQ ID NO:37 (encoding the INSP081 exon 19 polypeptide), SEQ ID NO:39 (encoding the INSP081 exon 20 polypeptide) and/or SEQ ID NO:41 (encoding the INSP081 polypeptide) or is a redundant equivalent or fragment of any one of these sequences.

The invention further provides that the purified nucleic acid molecule consists of the nucleic acid sequence as recited in SEQ ID NO:1 (encoding the INSP081 exon 1 polypeptide), SEQ ID NO:3 (encoding the INSP081 exon 2 polypeptide), SEQ ID NO:5 (encoding the INSP081 exon 3 polypeptide), SEQ ID NO:7 (encoding the INSP081 exon 4 polypeptide), SEQ ID NO:9 (encoding the INSP081 exon 5 polypeptide), SEQ ID NO:11 (encoding the INSP081 exon 6 polypeptide), SEQ ID NO:13 (encoding the INSP081 exon 7 polypeptide), SEQ ID NO:15 (encoding the INSP081 exon 8 polypeptide), SEQ ID NO:17 (encoding the INSP081 exon 9 polypeptide), SEQ ID NO:19 (encoding the INSP081 exon 10 polypeptide), SEQ ID NO:21 (encoding the INSP081 exon 11 polypeptide), SEQ ID NO:23 (encoding the INSP081 exon 12 polypeptide), SEQ ID NO:25 (encoding the INSP081 exon 13 polypeptide), SEQ ID NO:27 (encoding the INSP081 exon 14 polypeptide), SEQ ID NO:29 (encoding the INSP081 exon 15 polypeptide), SEQ ID NO:31 (encoding the INSP081 exon 16 polypeptide), SEQ ID NO:33 (encoding the INSP081 exon 17 polypeptide), SEQ ID NO:35 (encoding the INSP081 exon 18 polypeptide), SEQ ID NO:37 (encoding the INSP081 exon 19 polypeptide), SEQ ID NO:39 (encoding the INSP081 exon 20 polypeptide) and/or SEQ ID NO:41 (encoding the INSP081 polypeptide) or is a redundant equivalent or fragment of any one of these sequences.

In a second embodiment of the second aspect of the invention, the purified nucleic acid molecule comprises the nucleic acid sequence as recited in SEQ ID NO:43 (encoding the INSP082 exon 1 polypeptide), SEQ ID NO:45 (encoding the INSP082 exon 2 polypeptide), SEQ ID NO:47 (encoding the INSP082 exon 3 polypeptide), SEQ ID NO:49 (encoding the INSP082 exon 4 polypeptide), SEQ ID NO:51 (encoding the INSP082 exon 5 polypeptide), SEQ ID NO:53 (encoding the INSP082 exon 6 polypeptide), SEQ ID NO:55 (encoding the INSP082 exon 7 polypeptide), SEQ ID NO:57 (encoding the INSP082 exon 8 polypeptide), SEQ ID NO:59 (encoding the INSP082 exon 9 polypeptide), SEQ ID NO:61 (encoding the INSP082 exon 10 polypeptide), SEQ ID NO:63 (encoding the INSP082 exon 11 polypeptide), SEQ ID NO:65 (encoding the INSP082 exon 12 polypeptide), SEQ ID NO:67 (encoding the INSP082 exon 13 polypeptide), SEQ ID NO:69 (encoding the INSP082 exon 14 polypeptide), SEQ ID NO:71 (encoding the INSP082 exon 15 polypeptide), SEQ ID NO:73 (encoding the INSP082 exon 16 polypeptide), SEQ ID NO:75 (encoding the INSP082 exon 17 polypeptide), SEQ ID NO:77 (encoding the INSP082 exon 18 polypeptide), SEQ ID NO:79 (encoding the INSP082 exon 19 polypeptide), SEQ ID NO:81 (encoding the INSP082 exon 20 polypeptide), SEQ ID NO:83 (encoding the INSP082 exon 21 polypeptide), SEQ ID NO:85 (encoding the INSP082 exon 22 polypeptide), SEQ ID NO:87 (encoding the INSP082 exon 23 polypeptide), SEQ ID NO:89 (encoding the INSP082 exon 24 polypeptide), SEQ ID NO:91 (encoding the INSP082 exon 25 polypeptide), SEQ ID NO:93 (encoding the INSP082 exon 26 polypeptide), SEQ ID NO:95 (encoding the INSP082 exon 27 polypeptide), SEQ ID NO:97 (encoding the INSP082 exon 28 polypeptide) and/or SEQ ID NO:99 (encoding the INSP082 polypeptide) or is a redundant equivalent or fragment of any one of these sequences.

The invention further provides that the purified nucleic acid molecule consists of the nucleic acid sequence as recited in SEQ ID NO:43 (encoding the INSP082 exon 1 polypeptide), SEQ ID NO:45 (encoding the INSP082 exon 2 polypeptide), SEQ ID NO:47 (encoding the INSP082 exon 3 polypeptide), SEQ ID NO:49 (encoding the INSP082 exon 4 polypeptide), SEQ ID NO:51 (encoding the INSP082 exon 5 polypeptide), SEQ ID NO:53 (encoding the INSP082 exon 6 polypeptide), SEQ ID NO:55 (encoding the INSP082 exon 7 polypeptide), SEQ ID NO:57 (encoding the INSP082 exon 8 polypeptide), SEQ ID NO:59 (encoding the INSP082 exon 9 polypeptide), SEQ ID NO:61 (encoding the INSP082 exon 10 polypeptide), SEQ ID NO:63 (encoding the INSP082 exon 11 polypeptide), SEQ ID NO:65 (encoding the INSP082 exon 12 polypeptide), SEQ ID NO:67 (encoding the INSP082 exon 13 polypeptide), SEQ ID NO:69 (encoding the INSP082 exon 14 polypeptide), SEQ ID NO:71 (encoding the INSP082 exon 15 polypeptide), SEQ ID NO:73 (encoding the INSP082 exon 16 polypeptide), SEQ ID NO:75 (encoding the INSP082 exon 17 polypeptide), SEQ ID NO:77 (encoding the INSP082 exon 18 polypeptide), SEQ ID NO:79 (encoding the INSP082 exon 19 polypeptide), SEQ ID NO:81 (encoding the INSP082 exon 20 polypeptide), SEQ ID NO:83 (encoding the INSP082 exon 21 polypeptide), SEQ ID NO:85 (encoding the INSP082 exon 22 polypeptide), SEQ ID NO:87 (encoding the INSP082 exon 23 polypeptide), SEQ ID NO:89 (encoding the INSP082 exon 24 polypeptide), SEQ ID NO:91 (encoding the INSP082 exon 25 polypeptide), SEQ ID NO:93 (encoding the INSP082 exon 26 polypeptide), SEQ ID NO:95 (encoding the INSP082 exon 27 polypeptide), SEQ ID NO:97 (encoding the INSP082 exon 28 polypeptide) and/or SEQ ID NO:99 (encoding the INSP082 polypeptide) or is a redundant equivalent or fragment of any one of these sequences.

In a third embodiment of the second aspect of the invention, the purified nucleic acid molecule comprises the nucleic acid sequence as recited in SEQ ID NO:101 (encoding the INSP091 exon 1 polypeptide), SEQ ID NO:103 (encoding the INSP091 exon 2 polypeptide), SEQ ID NO:105 (encoding the INSP091 exon 3 polypeptide), SEQ ID NO:107 (encoding the INSP091 exon 4 polypeptide), SEQ ID NO:109 (encoding the INSP091 exon 5 polypeptide), SEQ ID NO:111 (encoding the INSP091 exon 6 polypeptide), SEQ ID NO:113 (encoding the INSP091 exon 7 polypeptide), SEQ ID NO:115 (encoding the INSP091 exon 8 polypeptide), SEQ ID NO:117 (encoding the INSP091 exon 9 polypeptide), SEQ ID NO:119 (encoding the INSP091 exon 10 polypeptide), SEQ ID NO:121 (encoding the INSP091 exon 11 polypeptide), SEQ ID NO:123 (encoding the INSP091 exon 12 polypeptide), SEQ ID NO:125 (encoding the INSP091 exon 13 polypeptide), SEQ ID NO:127 (encoding the INSP091 exon 14 polypeptide), SEQ ID NO:129 (encoding the INSP091 exon 15 polypeptide), SEQ ID NO:131 (encoding the INSP091 exon 16 polypeptide), SEQ ID NO:133 (encoding the INSP091 exon 17 polypeptide), SEQ ID NO:135 (encoding the INSP091 exon 18 polypeptide), SEQ ID NO:137 (encoding the INSP091 exon 19 polypeptide), SEQ ID NO:139 (encoding the INSP091 exon 20 polypeptide), SEQ ID NO:141 (encoding the INSP091 exon 21 polypeptide), SEQ ID NO:143 (encoding the INSP091 exon 22 polypeptide), SEQ ID NO:145 (encoding the INSP091 exon 23 polypeptide), SEQ ID NO:147 (encoding the INSP091 exon 24 polypeptide), SEQ ID NO:149 (encoding the INSP091 exon 25 polypeptide), SEQ ID NO:151 (encoding the INSP091 exon 26 polypeptide), SEQ ID NO:153 (encoding the INSP091 exon 27 polypeptide), SEQ ID NO:155 (encoding the INSP091 exon 28 polypeptide) and/or SEQ ID NO:157 (encoding the INSP091 polypeptide) or is a redundant equivalent or fragment of any one of these sequences.

The invention further provides that the purified nucleic acid molecule consists of the nucleic acid sequence as recited in SEQ ID NO:101 (encoding the INSP091 exon 1 polypeptide), SEQ ID NO:103 (encoding the INSP091 exon 2 polypeptide), SEQ ID NO:105 (encoding the INSP091 exon 3 polypeptide), SEQ ID NO:107 (encoding the INSP091 exon 4 polypeptide), SEQ ID NO:109 (encoding the INSP091 exon 5 polypeptide), SEQ ID NO:111 (encoding the INSP091 exon 6 polypeptide), SEQ ID NO:113 (encoding the INSP091 exon 7 polypeptide), SEQ ID NO:115 (encoding the INSP091 exon 8 polypeptide), SEQ ID NO:117 (encoding the INSP091 exon 9 polypeptide), SEQ ID NO:119 (encoding the INSP091 exon 10 polypeptide), SEQ ID NO:121 (encoding the INSP091 exon 11 polypeptide), SEQ ID NO:123 (encoding the INSP091 exon 12 polypeptide), SEQ ID NO:125 (encoding the INSP091 exon 13 polypeptide), SEQ ID NO:127 (encoding the INSP091 exon 14 polypeptide), SEQ ID NO:129 (encoding the INSP091 exon 15 polypeptide), SEQ ID NO:131 (encoding the INSP091 exon 16 polypeptide), SEQ ID NO:133 (encoding the INSP091 exon 17 polypeptide), SEQ ID NO:135 (encoding the INSP091 exon 18 polypeptide), SEQ ID NO:137 (encoding the INSP091 exon 19 polypeptide), SEQ ID NO:139 (encoding the INSP091 exon 20 polypeptide), SEQ ID NO:141 (encoding the INSP091 exon 21 polypeptide), SEQ ID NO:143 (encoding the INSP091 exon 22 polypeptide), SEQ ID NO:145 (encoding the INSP091 exon 23 polypeptide), SEQ ID NO:147 (encoding the INSP091 exon 24 polypeptide), SEQ ID NO:149 (encoding the INSP091 exon 25 polypeptide), SEQ ID NO:151 (encoding the INSP091 exon 26 polypeptide), SEQ ID NO:153 (encoding the INSP091 exon 27 polypeptide), SEQ ID NO:155 (encoding the INSP091 exon 28 polypeptide) and/or SEQ ID NO:157 (encoding the INSP091 polypeptide) or is a redundant equivalent or fragment of any one of these sequences.

Unigene cluster Hs.369523 is annotated as being weakly similar to STE20 protein kinase. Contained within this cluster are the human ESTs AI792963 (sourced from kidney and ovarian tissue), AI791391, AA865818, AI732997, AA977633 and BM546293 (sourced from ovarian tissue). These human ESTs are specifically excluded from the scope of this aspect of the invention. The BG622476 Human EST sequence is also specifically excluded from the scope of this aspect of the invention.

Murine mRNAs AB035267 and AB020741 are also specifically excluded from the scope of this aspect of the invention.

In a third aspect, the invention provides a purified nucleic acid molecule which hybridizes under high stringency conditions with a nucleic acid molecule of the second aspect of the invention.

In a fourth aspect, the invention provides a vector, such as an expression vector, that contains a nucleic acid molecule of the second or third aspect of the invention. I

n a fifth aspect, the invention provides a host cell transformed with a vector of the fourth aspect of the invention.

In a sixth aspect, the invention provides a ligand which binds specifically to members of the Germinal Center Kinase (GCK) subfamily of the STE20 family of protein kinases, preferably to a NIK-like kinase and more preferably to a NIK-like embryo specific kinase (NESK) of the first aspect of the invention. Preferably, the ligand inhibits the function of a polypeptide of the first aspect of the invention which is a member of the Germinal Center Kinase (GCK) subfamily of the STE20 family of protein kinases, preferably to a NIK-like kinase and more preferably to a NIK-like embryo specific kinase (NESK) of the first aspect of the invention. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonise) the level of expression of the gene or the activity of the polypeptide.

Importantly, the identification of the function of the INSP081, INSP082 and INSP091 polypeptides allows for the design of screening methods capable of identifying compounds that are effective in the treatment and/or diagnosis of disease. As used herein, the term “disease” also includes disorders. Ligands and compounds according to the sixth and seventh aspects of the invention may be identified using such methods. These methods are included as aspects of the present invention.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which members of the Germinal Center Kinase (GCK) subfamily of the STE20 family of protein kinases, preferably NIK-like kinases and more preferably NIK-like embryo specific kinases (NESK) are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions and in particular developmental disorders of late embryogenesis and neural tube defects such as spina bifida. These molecules may also be used in the manufacture of a medicament for the treatment of such disorders.

In a ninth aspect, the invention provides a method of diagnosing a disease or disorder in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease or disorder. Such a method will preferably be carried out in vitro. Similar methods may be used for monitoring the therapeutic treatment of disease or disorder in a patient, wherein altering the level of expression or activity of a polypeptide or nucleic acid molecule over the period of time towards a control level is indicative of regression of disease or disorder.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a Germinal Center Kinase (GCK), preferably as a NIK-like kinase and more preferably as a NIK-like embryo specific kinase (NESK). Suitable uses of the polypeptides of the invention as Germinal Center Kinases (GCK), preferably as NIK-like kinases and more preferably as NIK-like embryo specific kinases (NESK) include use as a regulator of cellular growth, metabolism or differentiation, use as part of a receptor/ligand pair and use as a diagnostic marker for a physiological or pathological condition.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease or disorder.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases or disorders in which the expression of a natural gene encoding a polypeptide of the first aspect of the invention, or in which the activity of a polypeptide of the first aspect of the invention, is lower in a diseased patient or a patient affected by a disorder when compared to the level of expression or activity in a healthy patient, the polypeptide, nucleic acid molecule, ligand or compound administered to the patient should be an agonist. Conversely, for diseases or disorders in which the expression of the natural gene or activity of the polypeptide is higher in a diseased patient or in a patient affected by a disorder when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease/disorders and may also be used in screening regimes for the identification of compounds that are effective in the treatment or diagnosis of such a disease/disorder.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attaclunent of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The fimctionally-equivalent polypeptides of the first aspect of the invention may be polypeptides that are homologous to the INSP081, INSP082 and INSP091 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP081, INSP082 and INSP091 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 30% identity between two polypeptides is considered to be an indication of functional equivalence. Preferably, functionally equivalent polypeptides of the first aspect of the invention have a degree of sequence identity with the INSP081, INSP082 or INSP091 polypeptides, or with active fragments thereof, of greater than 80%. More preferred polypeptides have degrees of identity of greater than 85%, 90%, 95%, 98% or 99%, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium™ search database may be used (see PCT application WO 01/69507) to identify polypeptides of presently-unknown function which, while having low sequence identity as compared to the INSP081, INSP082 and INSP091 polypeptides, are predicted to be members of the Germinal Center Kinase (GCK) subfamily of the STE20 family of protein kinases, preferably NIK-like kinases and more preferably NIK-like embryo specific kinases (NESK), by virtue of sharing significant structural homology with the INSP081, INSP082 and INSP091 polypeptide sequences. By “significant structural homology” is meant that the Inpharmatica Genome Threader predicts two proteins to share structural homology with a certainty of 10% and above.

The polypeptides of the first aspect of the invention also include fragments of the INSP081, INSP082 and INSP091 polypeptides and fragments of the functional equivalents of the INSP081, INSP082 and INSP091 polypeptides, provided that those fragments are members of the Germinal Center Kinase (GCK) subfamily of the STE20 family of protein kinases, preferably NIK-like kinases and more preferably NIK-like embryo specific kinases (NESK) or have an antigenic determinant in common with the INSP081, INSP082 and INSP091 polypeptides.

As used herein, the term “fragment” refers to a polypeptide having an amino acid sequence that is the same as part, but not all, of the amino acid sequence of the INSP081, INSP082 and INSP091 polypeptides or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the full length INSP081 polypeptides may comprise combinations of 2, 3, 4, 5, 6, 7, 8, 9, 10, 11, 12, 13, 14, 15, 16, 17, 18, 19 or 20 neighbouring exon sequences in the INSP081 polypeptide sequences (for example, they may consist of a fragment having the sequence given in exons 1 and 2, in exons 6, 7 and 8, in exons 10, 11, 12, 13, 14, 15 and 16 and so forth).

Fragments of the full length INSP082 polypeptides may comprise combinations of 2, 3, 4, 5, 6, 7, 8, 9, 10, 11, 12, 13, 14, 15, 16, 17, 18, 19, 20, 21, 22, 23, 24, 25, 26, 27 or 28 neighbouring exon sequences in the INSP082 polypeptide sequences.

Fragments of the full length INSP091 polypeptides may comprise combinations of 2, 3, 4, 5, 6, 7, 8, 9, 10, 11, 12, 13, 14, 15, 16, 17, 18, 19, 20, 21, 22, 23, 24, 25, 26, 27 or 28 neighbouring exon sequences in the INSP091 polypeptide sequences.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)₂ and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known members of the Germinal Center Kinase (GCK) subfamily of the STE20 family of protein kinases, such as NIK-like kinases and NIK-like embryo specific kinases (NESK).

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known members of the Germinal Center Kinase (GCK) subfamily of the STE20 family of protein kinases, such as NIK-like kinases and NIK-like embryo specific kinases (NESK).

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO: :156 and/or SEQ ID NO:158 and functionally equivalent polypeptides.

These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 and/or SEQ ID NO:158. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra)). A substantially homologous molecule will then compete for and inhibit the binding of a completely homologous molecule to the target molecule under various conditions of stringency, as taught in Wahl, G. M. and S. L. Berger (1987; Methods Enzymol. 152:399407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in

0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP081, INSP082 or INSP091 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INSP081, INSP082 or INSP091 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP081, INSP082 or INSP091 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP081, INSP082 or INSP091 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989,1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47 SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155 and SEQ ID NO:157) are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions. When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease/disorders. Such inappropriate expression may be of a temporal, spatial or quantitative nature.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, N.Y., Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al, Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chrornatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al, Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises: determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

Assays suitable for examining the biological activity of the INSP081 and INSP082 polypeptides include assays for kinase activity and MAP kinase pathway activation assays, as described in Nakano et al. JBC, (2000) 275, 27, 20533-20539.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease or disorder in a patient, comprising assessing the level of expression of a natural gene encoding a polypeptide according to the invention and comparing said level of expression to a control level, wherein a level that is different to said control level is indicative of disease. The method may comprise the steps of:
a)contacting a sample of tissue from the patient with a nucleic acid probe under stringent conditions that allow the formation of a hybrid complex between a nucleic acid molecule of the invention and the probe;
b)contacting a control sample with said probe under the same conditions used in step a);
c)and detecting the presence of hybrid complexes in said samples;
wherein detection of levels of the hybrid complex in the patient sample that differ from levels of the hybrid complex in the control sample is indicative of disease.

A further aspect of the invention comprises a diagnostic method comprising the steps of:
a)obtaining a tissue sample from a patient being tested for a disease;
b)isolating a nucleic acid molecule according to the invention from said tissue sample; and
c)diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCk. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 43974401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al).

In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:
(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in which members of the Germinal Center Kinase (GCK) subfamily of the STE20 family of protein kinases, preferably NIK-like kinases and more preferably NIK-like embryo specific kinases (NESK) are implicated.′ Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions and in particular developmental disorders of late embryogenesis and neural tube defects such as spina bifida.

Various aspects and embodiments of the present invention will now be described in more detail by way of example, with particular reference to the INSP081, INSP082 and INSP091 polypeptides.

It will be appreciated that modification of detail may be made without departing from the scope of the invention.

### BRIEF DESCRIPTION OF THE FIGURES

FIG. 1: Top ten results from BLAST against NCBI non-redundant database using SEQ ID NO:42 (INSP081 full protein sequence).

FIG. 2: Alignment generated by BLAST between SEQ ID NO:42 (INSP081 full protein sequence) and the top hit, (AB035267) Nck-interacting kinase-like embryo specific kinase from Mus. musculus.

FIG. 3: Top ten results from BLAST against NCBI non-redundant database using SEQ ID NO:100 (NSP082 full protein sequence).

FIG. 4: Alignment generated by BLAST between SEQ ID NO:100 (INSP082 full protein sequence) and the top hit, (AB035267) Nck-interacting kinase-like embryo specific kinase from Mus. musculus.

FIG. 5: Top ten results from BLAST against NCBI non-redundant database using SEQ ID NO:158 (INSP091 full protein sequence).

FIG. 6: Alignment generated by BLAST between SEQ ID NO:158 (INSP091 full protein sequence) and the top hit, (AB035267) Nck-interacting kinase-like embryo specific kinase from Mus. musculus.

FIG. 7: CLUSTAL alignment of INSP081, 082, 091 & published ZC4 coding sequences.

FIG. 8: Nucleotide sequence with translation of INSP082 PCR product cloned using primers INSP082-CP1 and INSP082-CP2.

FIG. 9: Nucleotide sequence with translation of INSP082 PCR product cloned using primers INSP082-CP3 and INSP082-CP4.

### EXAMPLES

### Example 1

### INSP081 Protein BLAST Results

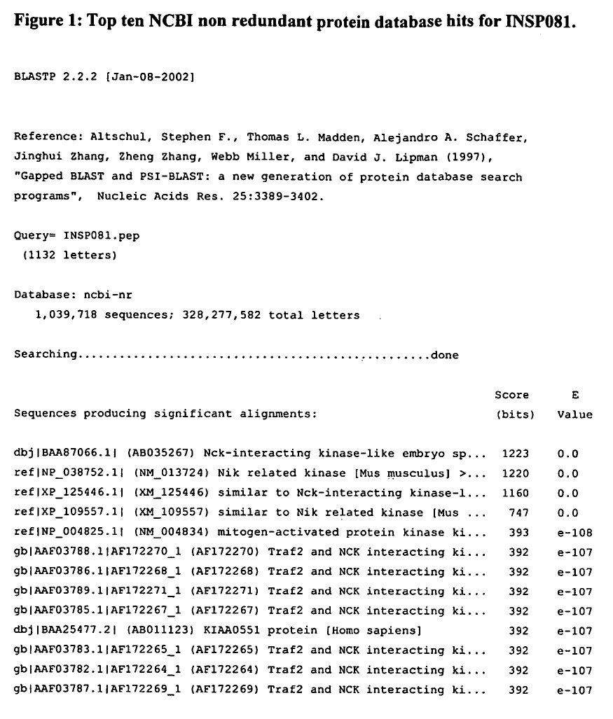

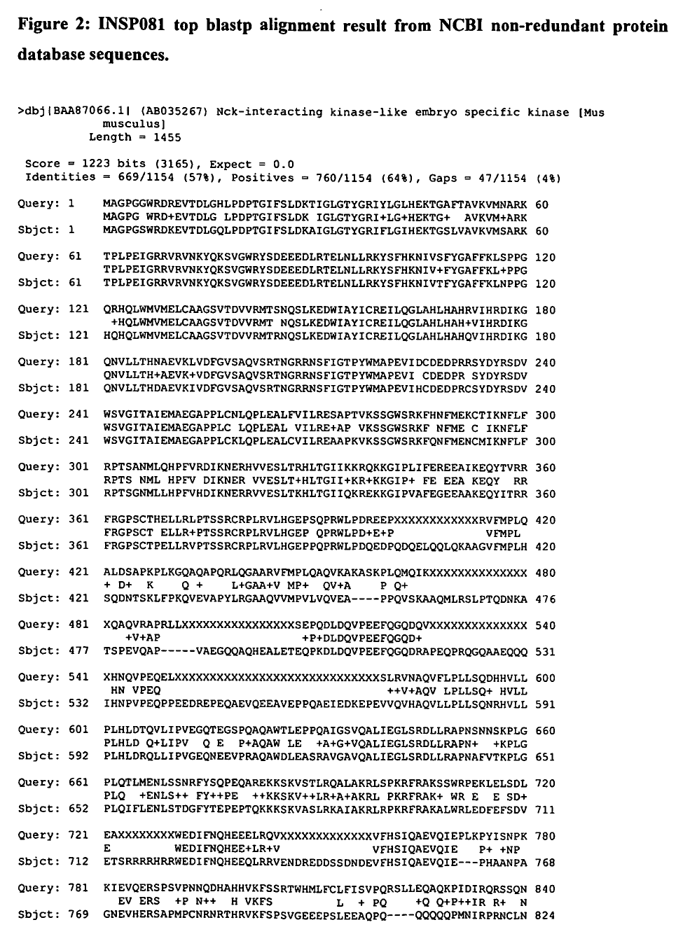

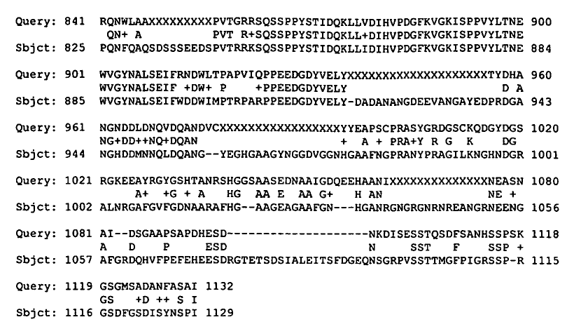

The INSP081 polypeptide sequence, shown in SEQ ID NO:42, was used as a BLAST query against the NCBI non-redundant sequence database. As can be seen in FIG. 1, the top hit is to Nck-interacting kinase-like embryo specific kinase (NESK) from Mus. musculus. INSP081 has 57% identity over its 1132 residues to the top hit. The majority of this identity occurs at the N-terminal of the protein. This is because INSP082 contains the NESK-like kinase domain at its N-terminal. The top hit has an expectation value of 0.

The closest human BLASTP homologue is the fifth top hit, (NM_{—}004834) mitogen-activated protein kinase kinase kinase kinase 4, HPK/GCK-like kinase. INSP081 has 58% identity to this human homologue over 326 of its residues. The closest human hit has an expectation value of e⁻¹⁰⁸.

The fact that all the top ten hits are from NESK or NIK-like kinases together with the expectation values given for these hits (the top four hits have expectation values of zero whilst the top ten hits all have an expectation value of e⁻¹⁰⁷ or less, which is extremely low), indicates that INSP081 is a member of the Germinal Center Kinase (GCK) subfamily of the STE20 family of protein kinases and is preferably a NIK-like kinase and is even more preferably a NIK-like embryo specific kinase (NESK).

### Example 2

### INSP082 Protein BLAST Results

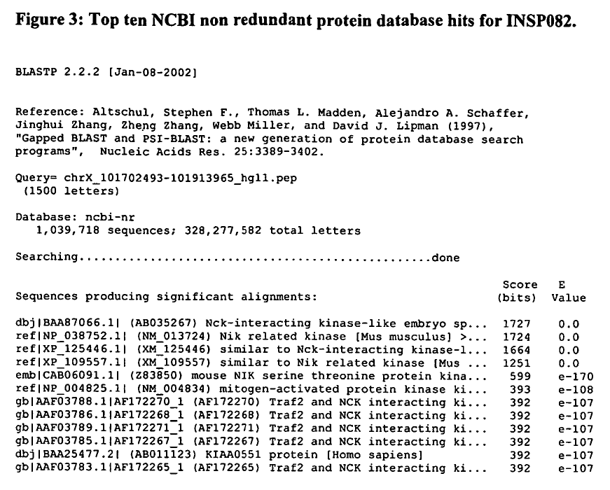

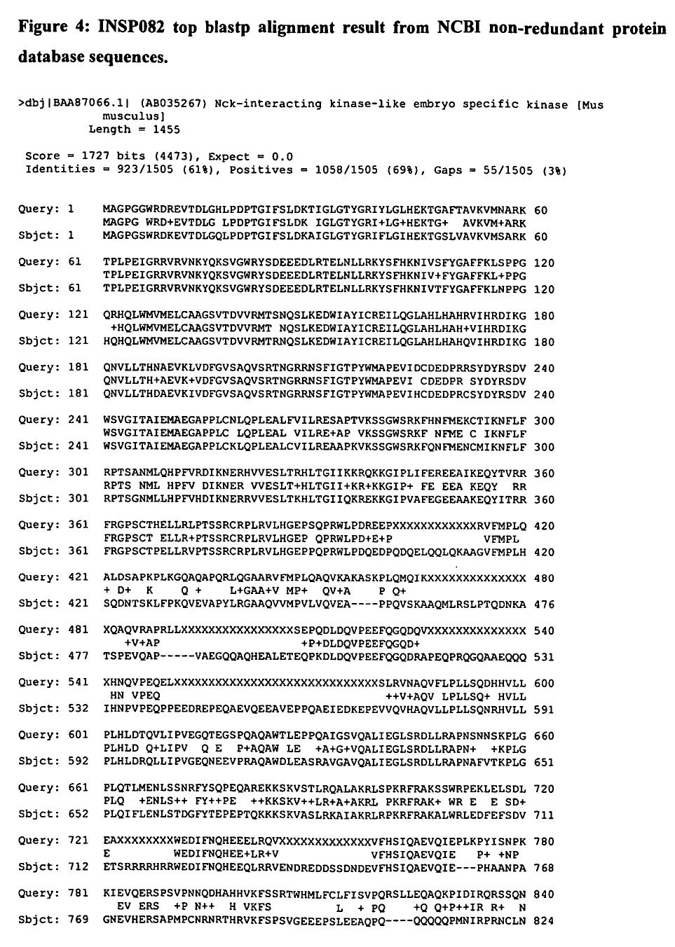

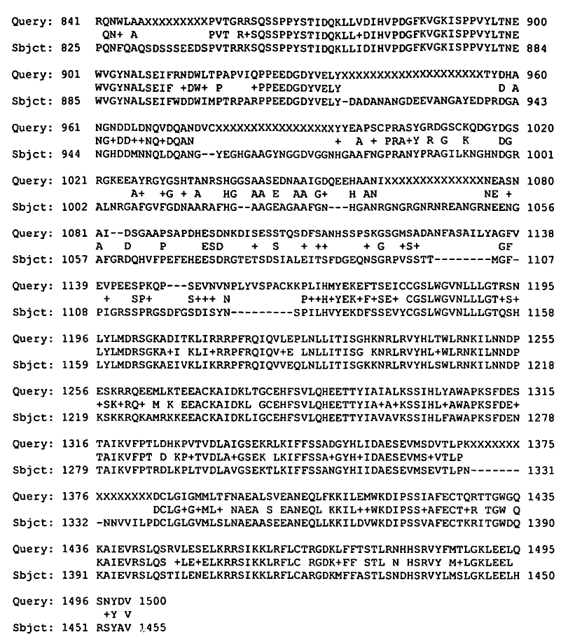

The INSP082 polypeptide sequence, shown in SEQ ID NO:100, was used as a BLAST query against the NCBI non-redundant sequence database. As can be seen in FIG. 3, the top hits is to Nck-interacting kinase-like embryo specific kinase (NESK) from Mus. musculus and hits two to ten are all from NESK or NIK-like kinases. The top four hits have expectation values of zero whilst the top ten hits all have an expectation value of e⁻¹⁰⁷ or less, which is extremely low. This therefore indicates that INSP082 is a member of the Germinal Center Kinase (GCK) subfamily of the STE20 family of protein kinases, preferably a NIK-like kinase and is even more preferably a NIK-like embryo specific kinase (NESK).

### Example 3

### INSP091 Protein BLAST Results

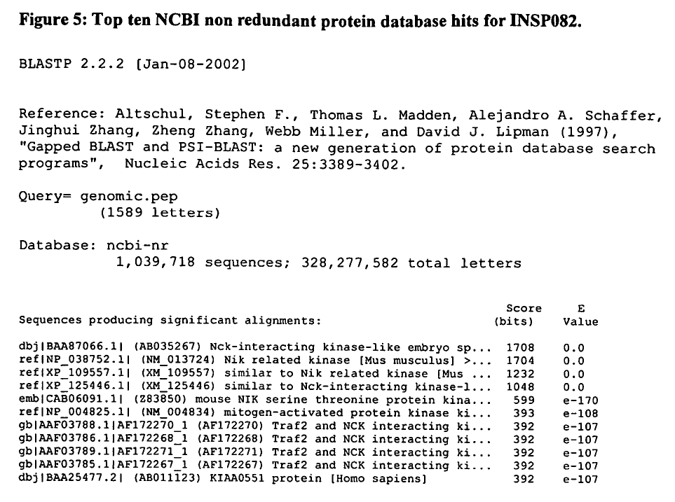

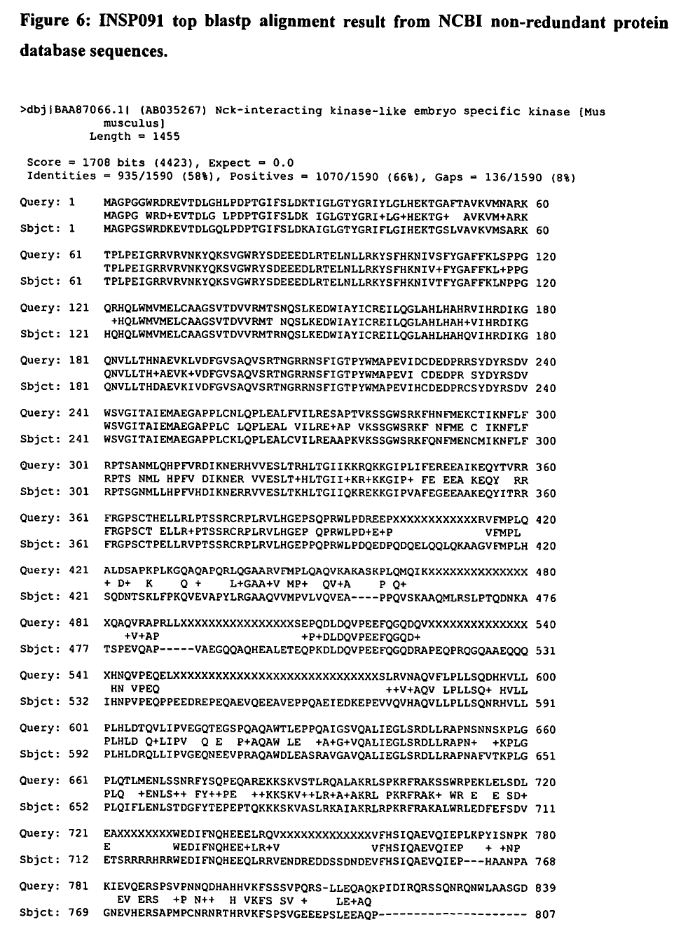

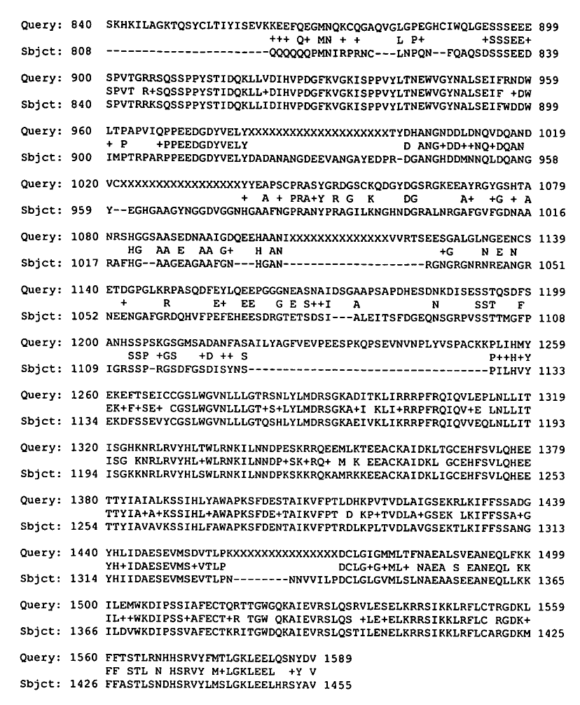

The INSP082 polypeptide sequence, shown in SEQ ID NO:158, was used as a BLAST query against the NCBI non-redundant sequence database. As can be seen in FIG. 5, the top hits is to Nck-interacting kinase-like embryo specific kinase (NESK) from Mus. musculus and hits two to ten are all from NESK or NIK-like kinases. The top four hits have expectation values of zero whilst the top ten hits all have an expectation value of e^{1 07} or less, which is extremely low. This therefore indicates that INSP091 is a member of the Germinal Center Kinase (GCK) subfamily of the STE20 family of protein kinases, preferably a NIK-like kinase and is even more preferably a NIK-like embryo specific kinase (NESK).

### Example 4

### Summary of Cloning Attempts of INSP081, INSP082 and INSP091

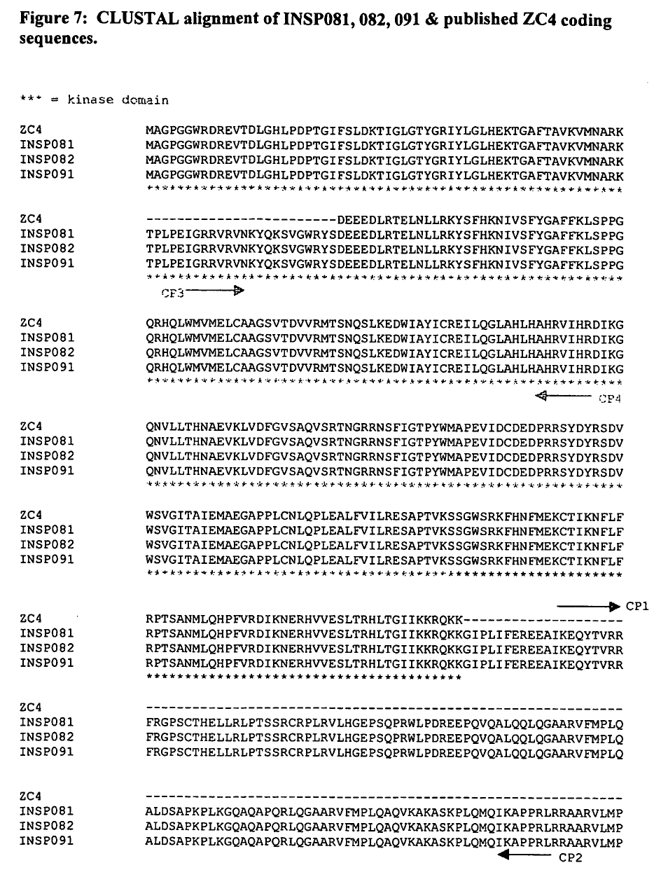

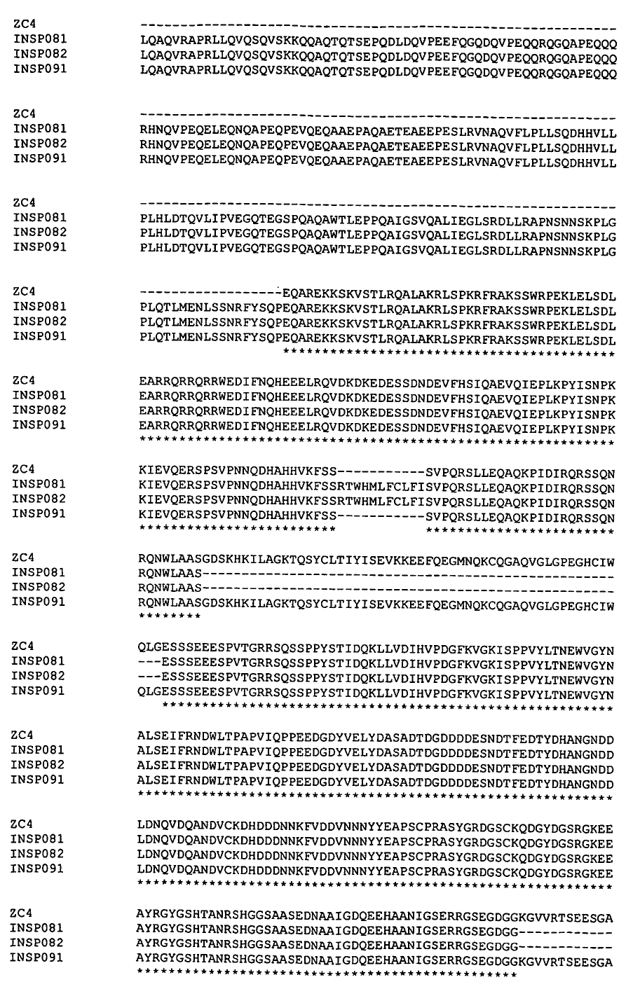

Two pairs of PCR amplification primers were designed, each intended to amplify a region of sequence which was common to all three of INSP081, INSP082 and INSP091. The first primer pair, called INSP082-CP1/-CP2, was designed to amplify a 355 bp region containing sequence from two exons and spanning 1058 to 1412 bp of the coding sequence of the INSP082 polypeptide. The second primer pair, called INSP082-CP3/-CP4, was designed to amplify a 332 bp region containing sequence from four exons covering 197 to 528 bp of the INSP082 coding sequence. Both primer pairs were designed to amplify sequence not present in the related published sequence ZC4 (see WO 99/53036) identified by homology searching in the NAGeneSeq database (see alignment below). Primers were designed using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (little or no none specific priming). The sequence of the primers is given in Table 1 and the positions of the primers are shown in FIG. 7.

TABLE 1
INSP082 cloning primers

| | |
|---|---|
| Primer | Sequence (5′-3′) |

| | |
|---|---|
| INSP082-CP1 | AGG AAC AGT ACA CCG TGA GA |
| INSP082-CP2 | AGT CGT GGA GGT GCC TTA AT |
| INSP082-CP3 | TAG GAA GGC GAG TGA GAG TG |
| INSP082-CP3 | CCG GTG AAT TAC TCG GTG TG |

cDNA pools were prepared as follows: First strand cDNA was prepared from a variety of normal human tissue total RNA samples (purchased from Clontech, Stratagene, Ambion, Biochain Institute and from in-house RNA samples) using Superscript II Rnase H⁻ Reverse Transcriptase (Invitrogen) according to the manufacturer's protocol. Oligo (dT)₁₅ primer (1 μl at 500 μg/ml, Promega), 2 μg human total RNA, 1 μl of 10 mM dNTP Mix (10 mM each dATP, dGTP, dCTP and dTTP at neutral pH) and sterile distilled water to a final volume of 12 μl were combined in a 1.5 ml Eppendorf tube, heated to 65 ° C. for 5 min and then chilled on ice. The contents were collected by brief centrifugation and 4 μl of 5× First-Strand Buffer, 2 μl of 0.1 M DTT, and 1 μl of RnaseOUT Recombinant Ribonuclease Inhibitor (40 units/μl, Invitrogen) were added. The contents of the tube were mixed gently and incubated at 42 ° C. for 2 min, then 1 μl (200 units) of SuperScript II enzyme was added and mixed gently by pipetting. The mixture was incubated at 42 ° C. for 50 min and then inactivated by heating at 70 ° C. for 15 min. To remove RNA complementary to the cDNA, 1 μl (2 units) of E. coli RNase H (Invitrogen) was added and the reaction mixture incubated at 37° C. for 20 min. The final reaction mix was diluted to give a total volume of 200 μl.

Equal volumes of five different cDNA samples were combined into pools. Five μl of each cDNA pool was used as a template for PCR in a 50 μl final reaction volume (corresponding to 1 μl or approximately 20 ng of each individual cDNA template).

PCR amplification was used to test the two primer pairs on the panel of cDNA pools. The PCR was performed in a final volume of 50 μl containing 1× AmpliTaq™ buffer, 200 μM dNTPs, 50 μmoles each of cloning primer, 2.5 units of AmpliTaq™ (Perkin Elmer) and 100 ng of each cDNA pool using an MJ Research DNA Engine, programmed as follows: 94° C., 2 min; 40 cycles of 94° C., 1 min, 54° C., 1 min, and 72° C., 1 min; folowed by 1 cycle at 72° C. for 7 min and a holding cycle at 4° C. The amplification products were visualized on 0.8% agarose gels in 1× TAE buffer (Invitrogen).

Both pairs identified a product only in pool PS15 which contained cDNA samples from human brain, heart, kidney, liver and lung. PCR products were purified from the gel using the Wizard PCR Preps DNA Purification System (Promega). The PCR product was eluted in 50 pi of sterile water and subcloned directly.

PCT products were subcloned into the topoisomerase 1 modified cloning vector (pCR4-TOPO) using the TOPO cloning kit purchased from the Invitrogen Corporation using the conditions specified by the manufacturer. Briefly, 4 μl of gel purified PCR product from the human cDNA amplification was incubated for 15 min at room temperature with1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of room temperature SOC media was added. Samples were incubated with shaking (220 rpm) for 1 h at 37° C. The transformation mixture was then plated on L-broth (LB) plates containing ampicillin (100 μg/ml) and incubated overnight at 37° C. Colonies containing inserts were identified by colony PCR.

Ampicillin resistant colonies were inoculated into 50 μl sterile water using a sterile toothpick. A 10 μl aliquot of the inoculum was then subjected to PCR in a total reaction volume of 20 μl as described above, except the primers used were T3 and T7. The cycling conditions were as follows:94° C., 2 min; 30 cycles of 94° C., 30 sec, 47° C., 30 sec and 72° C. for 1 min. Samples were then maintained at 4° C. (holding cycle) before further analysis. PCR reaction products were analyzed on 1% agarose gels in 1× TAE buffer.

Colonies which gave the expected PCR product size (355 bp cDNA or 332 bp cDNA+187 bp due to the multiple cloning site) were grown up overnight at 37° C. in 5 ml L-Broth (LB) containing ampicillin (100 μg/ml) with shaking at 220 rpm.

Miniprep plasmid DNA was prepared from the 5 ml culture using a Qiaprep Turbo 9600 robotic system (Qiagen) or Wizard Plus SV Minipreps kit (Promega cat. No. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with the T7 primer and T3 primer using the BigDye Terminator system (Applied Biosystems cat. No. 4390246) according to the manufacturer's instructions. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

TABLE 2
INSP082 sequencing primers

| | |
|---|---|
| Primer | Sequence (5′-3′) |

| | |
|---|---|
| T7 primer | TAA TAC GAC TCA CTA TAG G |
| T3 primer | ATT AAC CCT CAC TAA AGG |

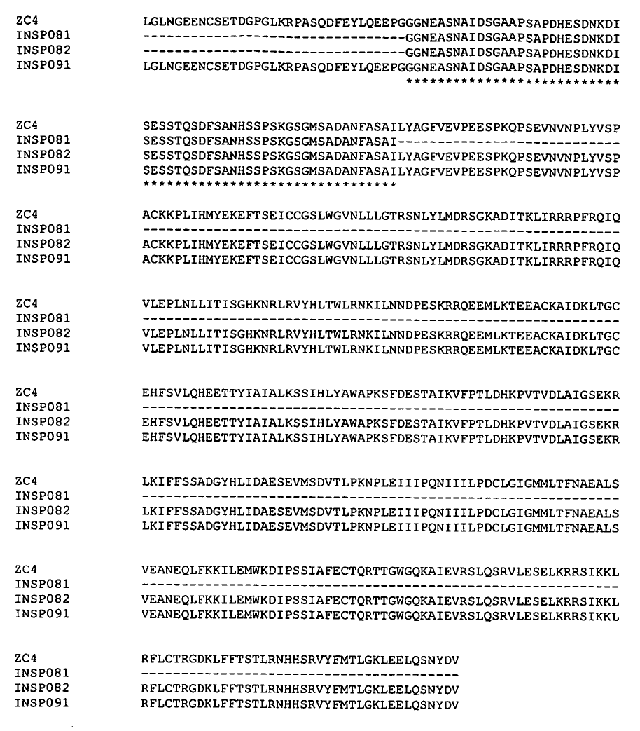

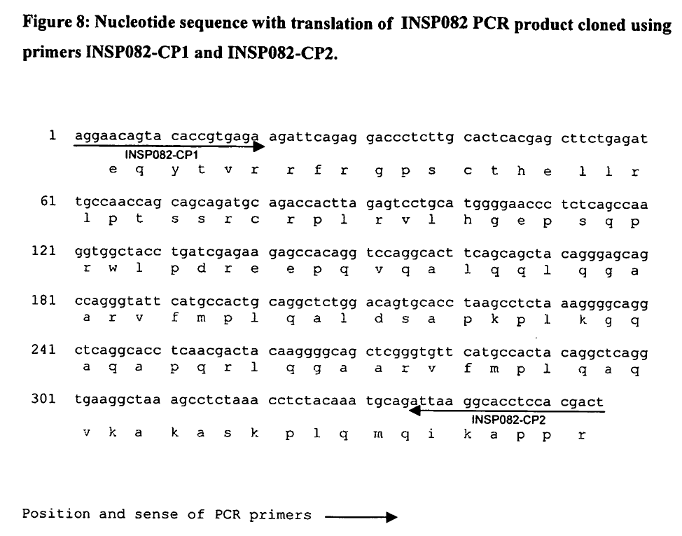

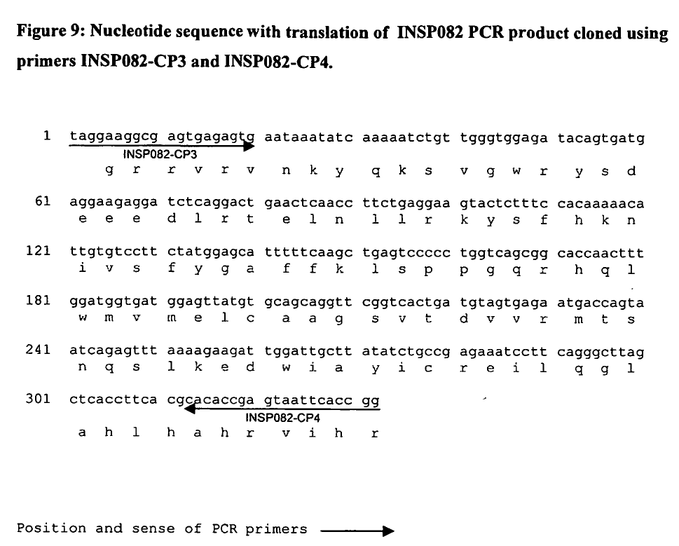

Sequence analysis identified one clone which contained a 100% match to the INSP082-CPI/INSP082-CP2 product sequence and one clone which contained a 100% match to the predicted INSP082-CP3/INSP082-CP4 product sequence. The sequences of these cloned cDNA fragments are shown in FIGS. 8 and 9 respectively.

Despite having identified potential tissue sources from these sequences, full length cloning of these predictions was not pursued because of the difficulties of cloning and expressing such long sequences.

### Example 5

### Expression and Purification of INSP081 INSP082 and INSP091

Further experiments may now be performed to determine the tissue distribution and expression levels of the INSP081, INSP082 and INSP091 polypeptides in vivo, on the basis of the nucleotide and amino acid sequence disclosed herein.

The presence of the transcripts for INSP081, INSP082 and INSP091 may be investigated by PCR of cDNA from different human tissues. The INSP081, INSP082 and INSP091 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a -ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase (-ve RT control). PCR reactions are set up for each tissue on the reverse transcribed RNA samples and the minus RT controls. INSP081, INSP082 and INSP091-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP081, INSP082 and INSP091 transcripts, not only those generated as described above. T

he tissue distribution pattern of the INSP081, INSP082 and INSP091 polypeptides will provide further useful information in relation to the function of those polypeptides.

In addition, further experiments may now be performed using the pEAK12d-INSP081-6HIS, pEAK12d-INSP082-6HIS and pEAK12d-INSP091-6HIS expression vectors.

Transfection of mammalian cell lines with these vectors may enable the high level expression of the INSP081, INSP082 and INSP091 proteins and thus enable the continued investigation of the functional characteristics of the INSP081, INSP082 and INSP091 polypeptides. The following material and methods are an example of those suitable in such experiments:

Cell Culture:

Human Embryonic Kidney 293 cells expressing the Epstein-Barr virus Nuclear Antigen (HEK293-EBNA, Invitrogen) are maintained in suspension in Ex-cell VPRO serum-free medium (seed stock, maintenance medium, JRH). Sixteen to 20 hours prior to transfection (Day-1), cells are seeded in 2× T225 flasks (50ml per flask in DMEM/F12 (1:1) containing 2% FBS seeding medium (JRH) at a density of 2×10⁵ cells/ml). The next day (transfection day 0) transfection takes place using the JetPEITM reagent (2 μl/μg of plasmid DNA, PolyPlus-transfection). For each flask, plasmid DNA is co-transfected with GFP (fluorescent reporter gene) DNA. The transfection mix is then added to the 2×T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 μl ) is kept for QC of the 6His-tagged protein (internal bioprocessing QC).

Scale-up batches may be produced by following the protocol called “PEI transfection of suspension cells”, referenced BP/PEI/HH/02/04, with PolyEthyleneImine from Polysciences as transfection agent.

Purification Process:

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amershain Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-His antibodies. The NuPAGE gel may be stained in a 0.1% Coomassie blue R250 staining solution (30% methanol, 10% acetic acid) at room temperature for Ih and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7mM KCl; 1.5 mM KH₂PO₄; 8 mM Na₂HPO₄; 0.1% Tween 20, pH 7.4 for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analysed.

For samples that showed detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP081, INSP082 and INSP091 polypeptides may be identified by immunoprecipitation combined with Western blotting and immunoprecipitation combined with mass spectroscopy.

1-54. (canceled)

55. A composition of matter:
(a) comprising an amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 or SEQ ID NO:158;
(b) consisting of an amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 or SEQ ID NO:158;
(c) that is a fragment of the amino acid sequence of (a) or (b) and which is a member of the Germinal Center Kinase (GCK) subfamily of the STE20 family of protein kinases, a NIK-like kinase, a NIK-like embryo specific kinase (NESK), or having an antigenic determinant in common with the polypeptide of (a) or (b);
(d) that is a functional equivalent of (a), (b) or (c);
(e) comprising an isolated polypeptide that is a functional equivalent of (a), (b) or (c) and is homologous to the amino acid sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 or SEQ ID NO:158 and is a member of the Germinal Center Kinase (GCK) subfamily of the STE20 family of protein kinases;
(f) comprising an isolated polypeptide which is a fragment or functional equivalent of a polypeptide as set forth in (a), (b), (c), (d), or (e), and that has greater than 80% sequence identity with the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156, SEQ ID NO:158 or with an active fragment thereof;
(g) which is a polypeptide fragment, or functional equivalent thereof, and has an antigenic determinant in common with a polypeptide which consists of 7 or more amino acid residues from the amino acid sequence recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, SEQ ID NO:150, SEQ ID NO:152, SEQ ID NO:154, SEQ ID NO:156 or SEQ ID NO:158;
(h) comprising a purified nucleic acid molecule which encodes a polypeptide according to (a), (b), (c), (d), (e), (f), or (g);
(i) comprising a purified nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47 SEQ ID NO:49, SEQ ID NO:5 1, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157 or is a redundant equivalent or fragment thereof;
(j) consisting of a purified nucleic acid sequence as recited in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47 SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, SEQ ID NO:149, SEQ ID NO:151, SEQ ID NO:153, SEQ ID NO:155, SEQ ID NO:157 or is a redundant equivalent or fragment thereof;
(k) comprising a purified nucleic acid molecule which hybridizes under high stringency conditions with a nucleic acid molecule according to any one of (h), (i) or 0);
(l) comprising a vector that comprises a nucleic acid molecule according to (h), (i), 0) or (k);
(m) comprising a host cell transformed with a vector according to (1);
(n) comprising a ligand which binds specifically to the Germinal Center Kinase (GCK) subfamily of the STE20 family of protein kinases;
(o) comprising a ligand that binds specifically to a polypeptide according to (a), (b), (c), (d), (e), (f) or (g);
(p) comprising a compound that increases or decreases the level of expression or activity of a polypeptide according to (a), (b), (c), (d), (e), (f) or (g);
(q) comprising a compound that binds to a polypeptide according to (a), (b), (c), (d), (e), (f) or (g) without inducing any of the biological effects of the polypeptide;
(r) comprising a pharmaceutical composition comprising a pharmaceutically acceptable carrier and (i) a polypeptide according to (a), (b), (c), (d), (e), (f) or (g); or (ii) a polynucleotide according to (h), (i), (j), (k) or (l);
(s) comprising a vaccine composition comprising (i) a polypeptide according to (a), (b), (c), (d), (e), (f) or (g); or (ii) a polynucleotide according to (h), (i), (j), (k) or (l);
(t) comprising a kit comprising a first container containing a nucleic acid probe that hybridizes under stringent conditions with a nucleic acid molecule according to (h), (i), (j), (k); a second container containing primers useful for amplifying said nucleic acid molecule; and instructions for using the probe and primers for facilitating the diagnosis of disease;
(u) comprising the kit of (t), further comprising a third container holding an agent for digesting un-hybridized RNA;
(v) comprising a kit comprising an array of nucleic acid molecules, at least one of which is a nucleic acid molecule according (h), (i), 0), (k);
(w) comprising a kit comprising one or more antibodies that bind to a polypeptide as recited in any one of (a), (b), (c), (d), (e), (f) or (g); and a reagent useful for the detection of a binding reaction between said antibody and said polypeptide; or
(x) comprising a transgenic or knockout non-human animal that has been transformed to express higher, lower or absent levels of a polypeptide according (a), (b), (c), (d), (e), (f) or (g).

56. The composition of matter according to claim 55(o), wherein said ligand is an antibody.

57. The composition of matter according to claim 55(q), wherein said compound is a natural or modified substrate, ligand, enzyme, receptor or structural or functional mimetic.

58. A method of diagnosing a disease in a patient comprising assessing the level of expression of a natural gene encoding a polypeptide or assessing the activity of a polypeptide according to claim 55 in tissue from said patient and comparing said level of expression or activity to a control level, wherein a level that is different to said control level is indicative of disease.

59. A method according to claim 58 that is carried out in vitro.

60. A method of using a composition of matter according to claim 55 for:
a) screening for a compound effective to treat disease, by contacting a non-human transgenic animal transformed to express higher, lower or absent levels of a polypeptide according to claim 55 with a candidate compound and determining the effect of the compound on the disease of the animal;
b) the identification of a compound that is effective in the treatment, diagnosis or both treatment and diagnosis of disease, comprising contacting a polypeptide or a polynucleotide according to claim 55 with one or more compounds suspected of possessing binding affinity for said polypeptide or nucleic acid molecule, and selecting a compound that binds specifically to said nucleic acid molecule or polypeptide;
c) for treating a disease in a patient comprising administering to the patient a polypeptide, a nucleic acid molecule, a vector, a host cell, a ligand, a compound or a pharmaceutical composition according to claim 55; or
d) for monitoring the therapeutic treatment of disease in a patient, comprising monitoring over a period of time the level of expression or activity of a polypeptide or a polynucleotide according to claim 55 in tissue from said patient, wherein altering said level of expression or activity over the period of time towards a control level is indicative of regression of said disease.

61. The method according to claim 60, wherein, for diseases in which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

62. The method according to claim 60, wherein, for diseases in which the expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.


SEQUENCE LISTING (164 sequences)

SEQ ID NO: 1 - DNA, 57 bp, Homo sapiens
atggcgggacctgggggctggagggacagggaggtcacggatctgggccacctgccg

SEQ ID NO: 2 - protein, 19 aa, Homo sapiens
MAGPGGWRDREVTDLGHLP

SEQ ID NO: 3 - DNA, 66 bp, Homo sapiens
gatccaactggaatattctcactagataaaaccattggccttggtacttatggcagaatctatttg

SEQ ID NO: 4 - protein, 22 aa, Homo sapiens
DPTGIFSLDKTIGLGTYGRIYL

SEQ ID NO: 5 - DNA, 57 bp, Homo sapiens
ggacttcatgagaagactggtgcatttacagctgttaaagtgatgaacgctcgtaag

SEQ ID NO: 6 - protein, 19 aa, Homo sapiens
GLHEKTGAFTAVKVMNARK

SEQ ID NO: 7 - DNA, 72 bp, Homo sapiens
acccctttacctgaaataggaaggcgagtgagagtgaataaatatcaaaaatctgttgggtggagatacagt

SEQ ID NO: 8 - protein, 24 aa, Homo sapiens
TPLPEIGRRVRVNKYQKSVGWRYS

SEQ ID NO: 9 - DNA, 126 bp, Homo sapiens
gatgaggaagaggatctcaggactgaactcaaccttctgaggaagtactctttccacaaaaacattgtgtccttctatggagcatttttcaagctgagtccccctggtcagcggcaccaactttgg

SEQ ID NO: 10 - protein, 42 aa, Homo sapiens
DEEEDLRTELNLLRKYSFHKNIVSFYGAFFKLSPPGQRHQLW

SEQ ID NO: 11 - DNA, 111 bp, Homo sapiens
atggtgatggagttatgtgcagcaggttcggtcactgatgtagtgagaatgaccagtaatcagagtttaaaagaagattggattgcttatatctgccgagaaatccttcag

SEQ ID NO: 12 - protein, 37 aa, Homo sapiens
MVMELCAAGSVTDVVRMTSNQSLKEDWIAYICREILQ

SEQ ID NO: 13 - DNA, 91 bp, Homo sapiens
ggcttagctcaccttcacgcacaccgagtaattcaccgggacatcaaaggtcagaatgtgctgctgactcataatgctgaagtaaaactgg

SEQ ID NO: 14 - protein, 31 aa, Homo sapiens
GLAHLHAHRVIHRDIKGQNVLLTHNAEVKLV

SEQ ID NO: 15 - DNA, 131 bp, Homo sapiens
ttgattttggagtgagtgcccaggtgagcagaactaatggaagaaggaatagtttcattgggacaccatactggatggcacctgaggtgattgactgtgatgaggacccaagacgctcctatgattacaga

SEQ ID NO: 16 - protein, 43 aa, Homo sapiens
DFGVSAQVSRTNGRRNSFIGTPYWMAPEVIDCDEDPRRSYDYR

SEQ ID NO: 17 - DNA, 55 bp, Homo sapiens
agtgatgtgtggtctgtgggaattactgccattgaaatggctgaaggagcccctc

SEQ ID NO: 18 - protein, 19 aa, Homo sapiens
SDVWSVGITAIEMAEGAPP

SEQ ID NO: 19 - DNA, 79 bp, Homo sapiens
ctctgtgtaaccttcaacccttggaagctctcttcgttattttgcgggaatctgctcccacagtcaaatccagcggatg

SEQ ID NO: 20 - protein, 26 aa, Homo sapiens
LCNLQPLEALFVILRESAPTVKSSGW

SEQ ID NO: 21 - DNA, 176 bp, Homo sapiens
gtcccgtaagttccacaatttcatggaaaagtgtacgataaaaaatttcctgtttcgtcctacttctgcaaacatgcttcaacacccatttgttcgggatataaaaaatgaacgacatgttgttgagtcattaacaaggcatcttactggaatcattaaaaaaagacagaaaaaag

SEQ ID NO: 22 - protein, 59 aa, Homo sapiens
SRKFHNFMEKCTIKNFLFRPTSANMLQHPFVRDIKNERHVVESLTRHLTGIIKKRQKKG

SEQ ID NO: 23 - DNA, 64 bp, Homo sapiens
gaatacctttgatctttgaaagagaagaagctattaaggaacagtacaccgtgagaagattcag

SEQ ID NO: 24 - protein, 21 aa, Homo sapiens
IPLIFEREEAIKEQYTVRRFR

SEQ ID NO: 25 - DNA, 1156 bp, Homo sapiens
aggaccctcttgcactcacgagcttctgagattgccaaccagcagcagatgcagaccacttagagtcctgcatggggaaccctctcagccaaggtggctacctgatcgagaagagccacaggtccaggcacttcagcagctacagggagcagccagggtattcatgccactgcaggctctggacagtgcacctaagcctctaaaggggcaggctcaggcacctcaacgactacaaggggcagctcgggtgttcatgccactacaggctcaggtgaaggctaaagcctctaaacctctacaaatgcagattaaggcacctccacgactacggagggcagccagggtgctcatgccactacaggcacaggttagggcacctaggcttctgcaggtacagtcccaggtatccaaaaagcagcaggcccagacccagacatcagaaccacaagatttggaccaggtaccagaggaatttcagggtcaagatcaggtacccgaacaacaaaggcagggccaggcccctgaacaacagcagaggcacaaccaggtgcctgaacaagagctggagcagaaccaggcacctgaacagccagaggtacaggaacaggctgccgagcctgcacaggcagagactgaggcagaggaacctgagtcattacgagtaaatgcccaggtatttctgcccctgctatcacaagatcaccatgtgctgttgccactacatttggatactcaggtgctcattccagtagaggggcaaactgaaggatcacctcaggcacaggcttggacactagaacccccacaggcaattggctcagttcaagcactgatagagggactatcaagagacttgcttcgggcaccaaactcaaataactcaaagccacttggtccgttgcaaaccctgatggaaaatctgtcatcaaataggttttactcacaaccagaacaggcacgggagaaaaaatcaaaagtttctactctgaggcaagcactggcaaaaagactatcaccaaagaggttcagggcaaagtcatcatggagacctgaaaagcttgaactctcggatttagaagcccgcaggcaaaggcgccaacgcagatgggaagatatctttaatcagcatgaggaagaattgagacaagttgataaa

SEQ ID NO: 26 - protein, 385 aa, Homo sapiens
GPSCTHELLRLPTSSRCRPLRVLHGEPSQPRWLPDREEPQVQALQQLQGAARVFMPLQALDSAPKPLKGQAQAPQRLQGAARVFMPLQAQVKAKASKPLQMQIKAPPRLRRAARVLMPLQAQVRAPRLLQVQSQVSKKQQAQTQTSEPQDLDQVPEEFQGQDQVPEQQRQGQAPEQQQRHNQVPEQELEQNQAPEQPEVQEQAAEPAQAETEAEEPESLRVNAQVFLPLLSQDHHVLLPLHLDTQVLIPVEGQTEGSPQAQAWTLEPPQAIGSVQALIEGLSRDLLRAPNSNNSKPLGPLQTLMENLSSNRFYSQPEQAREKKSKVSTLRQALAKRLSPKRFRAKSSWRPEKLELSDLEARRQRRQRRWEDIFNQHEEELRQVDK

SEQ ID NO: 27 - DNA, 108 bp, Homo sapiens
gacaaagaagatgaatcatcagacaatgatgaagtatttcattcgattcaggctgaagtccagatagagccattgaagccatacatttcaaatcctaaaaaaattgag

SEQ ID NO: 28 - protein, 36 aa, Homo sapiens
DKEDESSDNDEVFHSIQAEVQIEPLKPYISNPKKIE

SEQ ID NO: 29 - DNA, 68 bp, Homo sapiens
gttcaagagagatctccttctgtgcctaacaaccaggatcatgcacatcatgtcaagttctcttcaag

SEQ ID NO: 30 - protein, 23 aa, Homo sapiens
VQERSPSVPNNQDHAHHVKFSSR

SEQ ID NO: 31 - DNA, 128 bp, Homo sapiens
gacatggcacatgcttttctgtcttttcattagcgttcctcagcggtctcttttggaacaagctcagaagcccattgacatcagacaaaggagttcgcaaaatcgtcaaaattggctggcagcatcag

SEQ ID NO: 32 - protein, 43 aa, Homo sapiens
TWHMLFCLFISVPQRSLLEQAQKPIDIRQRSSQNRQNWLAASE

SEQ ID NO: 33 - DNA, 98 bp, Homo sapiens
aatcttcttctgaggaagaaagtcctgtgactggaaggaggtctcagtcatcaccaccttattctactattgatcagaagttgctggttgacatccat

SEQ ID NO: 34 - protein, 32 aa, Homo sapiens
SSSEEESPVTGRRSQSSPPYSTIDQKLLVDIH

SEQ ID NO: 35 - DNA, 365 bp, Homo sapiens
gttccagatggatttaaagtaggaaaaatatcaccccctgtatacttgacaaacgaatgggtaggctataatgcactctctgaaatcttccggaatgattggttaactccggcacctgtcattcagccacctgaagaggatggtgattatgttgaactctatgatgccagtgctgatactgatggtgatgatgatgatgagtctaatgatacttttgaagatacctatgatcatgccaatggcaatgatgacttggataaccaggttgatcaggctaatgatgtttgtaaagaccatgatgatgacaacaataagtttgttgatgatgtaaataataattattatgaggcgcctagttgtccaag

SEQ ID NO: 36 - protein, 122 aa, Homo sapiens
VPDGFKVGKISPPVYLTNEWVGYNALSEIFRNDWLTPAPVIQPPEEDGDYVELYDASADTDGDDDDESNDTFEDTYDHANGNDDLDNQVDQANDVCKDHDDDNNKFVDDVNNNYYEAPSCPR

SEQ ID NO: 37 - DNA, 209 bp, Homo sapiens
ggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggag

SEQ ID NO: 38 - protein, 70 aa, Homo sapiens
ASYGRDGSCKQDGYDGSRGKEEAYRGYGSHTANRSHGGSAASEDNAAIGDQEEHAANIGSERRGSEGDGG

SEQ ID NO: 39 - DNA, 182 bp, Homo sapiens
gtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatctaa

SEQ ID NO: 40 - protein, 59 aa, Homo sapiens
GGNEASNAIDSGAAPSAPDHESDNKDISESSTQSDFSANHSSPSKGSGMSADANFASAI

SEQ ID NO: 41 - DNA, 3396 bp, Homo sapiens
atggcgggacctgggggctggagggacagggaggtcacggatctgggccacctgccggatccaactggaatattctcactagataaaaccattggccttggtacttatggcagaatctatttgggacttcatgagaagactggtgcatttacagctgttaaagtgatgaacgctcgtaagacccctttacctgaaataggaaggcgagtgagagtgaataaatatcaaaaatctgttgggtggagatacagtgatgaggaagaggatctcaggactgaactcaaccttctgaggaagtactctttccacaaaaacattgtgtccttctatggagcatttttcaagctgagtccccctggtcagcggcaccaactttggatggtgatggagttatgtgcagcaggttcggtcactgatgtagtgagaatgaccagtaatcagagtttaaaagaagattggattgcttatatctgccgagaaatccttcagggcttagctcaccttcacgcacaccgagtaattcaccgggacatcaaaggtcagaatgtgctgctgactcataatgctgaagtaaaactggttgattttggagtgagtgcccaggtgagcagaactaatggaagaaggaatagtttcattgggacaccatactggatggcacctgaggtgattgactgtgatgaggacccaagacgctcctatgattacagaagtgatgtgtggtctgtgggaattactgccattgaaatggctgaaggagcccctcctctgtgtaaccttcaacccttggaagctctcttcgttattttgcgggaatctgctcccacagtcaaatccagcggatggtcccgtaagttccacaatttcatggaaaagtgtacgataaaaaatttcctgtttcgtcctacttctgcaaacatgcttcaacacccatttgttcgggatataaaaaatgaacgacatgttgttgagtcattaacaaggcatcttactggaatcattaaaaaaagacagaaaaaaggaatacctttgatctttgaaagagaagaagctattaaggaacagtacaccgtgagaagattcagaggaccctcttgcactcacgagcttctgagattgccaaccagcagcagatgcagaccacttagagtcctgcatggggaaccctctcagccaaggtggctacctgatcgagaagagccacaggtccaggcacttcagcagctacagggagcagccagggtattcatgccactgcaggctctggacagtgcacctaagcctctaaaggggcaggctcaggcacctcaacgactacaaggggcagctcgggtgttcatgccactacaggctcaggtgaaggctaaagcctctaaacctctacaaatgcagattaaggcacctccacgactacggagggcagccagggtgctcatgccactacaggcacaggttagggcacctaggcttctgcaggtacagtcccaggtatccaaaaagcagcaggcccagacccagacatcagaaccacaagatttggaccaggtaccagaggaatttcagggtcaagatcaggtacccgaacaacaaaggcagggccaggcccctgaacaacagcagaggcacaaccaggtgcctgaacaagagctggagcagaaccaggcacctgaacagccagaggtacaggaacaggctgccgagcctgcacaggcagagactgaggcagaggaacctgagtcattacgagtaaatgcccaggtatttctgcccctgctatcacaagatcaccatgtgctgttgccactacatttggatactcaggtgctcattccagtagaggggcaaactgaaggatcacctcaggcacaggcttggacactagaacccccacaggcaattggctcagttcaagcactgatagagggactatcaagagacttgcttcgggcaccaaactcaaataactcaaagccacttggtccgttgcaaaccctgatggaaaatctgtcatcaaataggttttactcacaaccagaacaggcacgggagaaaaaatcaaaagtttctactctgaggcaagcactggcaaaaagactatcaccaaagaggttcagggcaaagtcatcatggagacctgaaaagcttgaactctcggatttagaagcccgcaggcaaaggcgccaacgcagatgggaagatatctttaatcagcatgaggaagaattgagacaagttgataaagacaaagaagatgaatcatcagacaatgatgaagtatttcattcgattcaggctgaagtccagatagagccattgaagccatacatttcaaatcctaaaaaaattgaggttcaagagagatctccttctgtgcctaacaaccaggatcatgcacatcatgtcaagttctcttcaaggacatggcacatgcttttctgtcttttcattagcgttcctcagcggtctcttttggaacaagctcagaagcccattgacatcagacaaaggagttcgcaaaatcgtcaaaattggctggcagcatcagaatcttcttctgaggaagaaagtcctgtgactggaaggaggtctcagtcatcaccaccttattctactattgatcagaagttgctggttgacatccatgttccagatggatttaaagtaggaaaaatatcaccccctgtatacttgacaaacgaatgggtaggctataatgcactctctgaaatcttccggaatgattggttaactccggcacctgtcattcagccacctgaagaggatggtgattatgttgaactctatgatgccagtgctgatactgatggtgatgatgatgatgagtctaatgatacttttgaagatacctatgatcatgccaatggcaatgatgacttggataaccaggttgatcaggctaatgatgtttgtaaagaccatgatgatgacaacaataagtttgttgatgatgtaaataataattattatgaggcgcctagttgtccaagggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggaggtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatc

SEQ ID NO: 42 - protein, 1132 aa, Homo sapiens
MAGPGGWRDREVTDLGHLPDPTGIFSLDKTIGLGTYGRIYLGLHEKTGAFTAVKVMNARKTPLPEIGRRVRVNKYQKSVGWRYSDEEEDLRTELNLLRKYSFHKNIVSFYGAFFKLSPPGQRHQLWMVMELCAAGSVTDVVRMTSNQSLKEDWIAYICREILQGLAHLHAHRVIHRDIKGQNVLLTHNAEVKLVDFGVSAQVSRTNGRRNSFIGTPYWMAPEVIDCDEDPRRSYDYRSDVWSVGITAIEMAEGAPPLCNLQPLEALFVILRESAPTVKSSGWSRKFHNFMEKCTIKNFLFRPTSANMLQHPFVRDIKNERHVVESLTRHLTGIIKKRQKKGIPLIFEREEAIKEQYTVRRFRGPSCTHELLRLPTSSRCRPLRVLHGEPSQPRWLPDREEPQVQALQQLQGAARVFMPLQALDSAPKPLKGQAQAPQRLQGAARVFMPLQAQVKAKASKPLQMQIKAPPRLRRAARVLMPLQAQVRAPRLLQVQSQVSKKQQAQTQTSEPQDLDQVPEEFQGQDQVPEQQRQGQAPEQQQRHNQVPEQELEQNQAPEQPEVQEQAAEPAQAETEAEEPESLRVNAQVFLPLLSQDHHVLLPLHLDTQVLIPVEGQTEGSPQAQAWTLEPPQAIGSVQALIEGLSRDLLRAPNSNNSKPLGPLQTLMENLSSNRFYSQPEQAREKKSKVSTLRQALAKRLSPKRFRAKSSWRPEKLELSDLEARRQRRQRRWEDIFNQHEEELRQVDKDKEDESSDNDEVFHSIQAEVQIEPLKPYISNPKKIEVQERSPSVPNNQDHAHHVKFSSRTWHMLFCLFISVPQRSLLEQAQKPIDIRQRSSQNRQNWLAASESSSEEESPVTGRRSQSSPPYSTIDQKLLVDIHVPDGFKVGKISPPVYLTNEWVGYNALSEIFRNDWLTPAPVIQPPEEDGDYVELYDASADTDGDDDDESNDTFEDTYDHANGNDDLDNQVDQANDVCKDHDDDNNKFVDDVNNNYYEAPSCPRASYGRDGSCKQDGYDGSRGKEEAYRGYGSHTANRSHGGSAASEDNAAIGDQEEHAANIGSERRGSEGDGGGGNEASNAIDSGAAPSAPDHESDNKDISESSTQSDFSANHSSPSKGSGMSADANFASAI

SEQ ID NO: 43 - DNA, 57 bp, Homo sapiens; identical to SEQ ID NO: 1

SEQ ID NO: 44 - protein, 19 aa, Homo sapiens
MAGPGGWRDREVTDLGHLP

SEQ ID NO: 45 - DNA, 66 bp, Homo sapiens; identical to SEQ ID NO: 3

SEQ ID NO: 46 - protein, 22 aa, Homo sapiens
DPTGIFSLDKTIGLGTYGRIYL

SEQ ID NO: 47 - DNA, 57 bp, Homo sapiens; identical to SEQ ID NO: 5

SEQ ID NO: 48 - protein, 19 aa, Homo sapiens
GLHEKTGAFTAVKVMNARK

SEQ ID NO: 49 - DNA, 72 bp, Homo sapiens; identical to SEQ ID NO: 7

SEQ ID NO: 50 - protein, 24 aa, Homo sapiens
TPLPEIGRRVRVNKYQKSVGWRYS

SEQ ID NO: 51 - DNA, 126 bp, Homo sapiens; identical to SEQ ID NO: 9

SEQ ID NO: 52 - protein, 42 aa, Homo sapiens; identical to SEQ ID NO: 10

SEQ ID NO: 53 - DNA, 111 bp, Homo sapiens; identical to SEQ ID NO: 11

SEQ ID NO: 54 - protein, 37 aa, Homo sapiens; identical to SEQ ID NO: 12

SEQ ID NO: 55 - DNA, 91 bp, Homo sapiens; identical to SEQ ID NO: 13

SEQ ID NO: 56 - protein, 31 aa, Homo sapiens; identical to SEQ ID NO: 14

SEQ ID NO: 57 - DNA, 131 bp, Homo sapiens; identical to SEQ ID NO: 15

SEQ ID NO: 58 - protein, 43 aa, Homo sapiens; identical to SEQ ID NO: 16

SEQ ID NO: 59 - DNA, 55 bp, Homo sapiens; identical to SEQ ID NO: 17

SEQ ID NO: 60 - protein, 19 aa, Homo sapiens
SDVWSVGITAIEMAEGAPP

SEQ ID NO: 61 - DNA, 79 bp, Homo sapiens; identical to SEQ ID NO: 19

SEQ ID NO: 62 - protein, 26 aa, Homo sapiens
LCNLQPLEALFVILRESAPTVKSSGW

SEQ ID NO: 63 - DNA, 176 bp, Homo sapiens; identical to SEQ ID NO: 21

SEQ ID NO: 64 - protein, 59 aa, Homo sapiens; identical to SEQ ID NO: 22

SEQ ID NO: 65 - DNA, 64 bp, Homo sapiens; identical to SEQ ID NO: 23

SEQ ID NO: 66 - protein, 21 aa, Homo sapiens
IPLIFEREEAIKEQYTVRRFR

SEQ ID NO: 67 - DNA, 1156 bp, Homo sapiens; identical to SEQ ID NO: 25

SEQ ID NO: 68 - protein, 385 aa, Homo sapiens; identical to SEQ ID NO: 26

SEQ ID NO: 69 - DNA, 108 bp, Homo sapiens; identical to SEQ ID NO: 27

SEQ ID NO: 70 - protein, 36 aa, Homo sapiens; identical to SEQ ID NO: 28

SEQ ID NO: 71 - DNA, 68 bp, Homo sapiens; identical to SEQ ID NO: 29

SEQ ID NO: 72 - protein, 23 aa, Homo sapiens
VQERSPSVPNNQDHAHHVKFSSR

SEQ ID NO: 73 - DNA, 128 bp, Homo sapiens; identical to SEQ ID NO: 31

SEQ ID NO: 74 - protein, 43 aa, Homo sapiens; identical to SEQ ID NO: 32

SEQ ID NO: 75 - DNA, 98 bp, Homo sapiens; identical to SEQ ID NO: 33

SEQ ID NO: 76 - protein, 32 aa, Homo sapiens; identical to SEQ ID NO: 34

SEQ ID NO: 77 - DNA, 365 bp, Homo sapiens; identical to SEQ ID NO: 35

SEQ ID NO: 78 - protein, 122 aa, Homo sapiens; identical to SEQ ID NO: 36

SEQ ID NO: 79 - DNA, 209 bp, Homo sapiens; identical to SEQ ID NO: 37

SEQ ID NO: 80 - protein, 70 aa, Homo sapiens; identical to SEQ ID NO: 38

SEQ ID NO: 81 - DNA, 180 bp, Homo sapiens
gtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatct

SEQ ID NO: 82 - protein, 60 aa, Homo sapiens
GGNEASNAIDSGAAPSAPDHESDNKDISESSTQSDFSANHSSPSKGSGMSADANFASAIL

SEQ ID NO: 83 - DNA, 156 bp, Homo sapiens
tatacgctggattcgtagaagtacctgaggaatcacctaagcaaccctctgaagtcaatgttaacccactctatgtctctcctgcatgtaaaaaaccactaatccacatgtatgaaaaggagttcacttctgagatctgctgtggttctttgtggg

SEQ ID NO: 84 - protein, 52 aa, Homo sapiens
YAGFVEVPEESPKQPSEVNVNPLYVSPACKKPLIHMYEKEFTSEICCGSLWG

SEQ ID NO: 85 - DNA, 144 bp, Homo sapiens
gagtcaatttgctgttgggaacccgatctaatctatatctgatggacagaagtggaaaggctgacattactaaacttataaggcgaagaccattccgccagattcaagtcttagagccactcaatttgctgattaccatctcag

SEQ ID NO: 86 - protein, 48 aa, Homo sapiens
VNLLLGTRSNLYLMDRSGKADITKLIRRRPFRQIQVLEPLNLLITISG

SEQ ID NO: 87 - DNA, 159 bp, Homo sapiens
gtcataagaacagacttcgggtgtatcatctgacctggttgaggaacaagattttgaataatgatccagaaagtaaaagaaggcaagaagaaatgctgaagacagaggaagcctgcaaagctattgataagttaacaggctgtgaacacttcagtgtcc

SEQ ID NO: 88 - protein, 53 aa, Homo sapiens
HKNRLRVYHLTWLRNKILNNDPESKRRQEEMLKTEEACKAIDKLTGCEHFSVL

SEQ ID NO: 89 - DNA, 101 bp, Homo sapiens
tccaacatgaagaaacaacatatattgcaattgctttgaaatcatcaattcacctttatgcatgggcaccaaagtcctttgatgaaagcactgctattaaa

SEQ ID NO: 90 - protein, 33 aa, Homo sapiens
QHEETTYIAIALKSSIHLYAWAPKSFDESTAIK

SEQ ID NO: 91 - DNA, 150 bp, Homo sapiens
gtatttccaacacttgatcataagccagtgacagttgacctggctattggttctgaaaaaagactaaagattttcttcagctcagcagatggatatcacctcatcgatgcagaatctgaggttatgtctgatgtgaccctgccaaagaat

SEQ ID NO: 92 - protein, 50 aa, Homo sapiens
VFPTLDHKPVTVDLAIGSEKRLKIFFSSADGYHLIDAESEVMSDVTLPKN

SEQ ID NO: 93 - DNA, 160 bp, Homo sapiens
cccctggaaatcattataccacagaatatcatcattttacctgattgcttgggaattggcatgatgctcaccttcaatgctgaagccctctctgtggaagcaaatgaacaactcttcaagaagatccttgaaatgtggaaagacataccatcttctatag

SEQ ID NO: 94 - protein, 54 aa, Homo sapiens
PLEIIIPQNIIILPDCLGIGMMLTFNAEALSVEANEQLFKKILEMWKDIPSSIA

SEQ ID NO: 95 - DNA, 140 bp, Homo sapiens
cttttgaatgtacacagcgaaccacaggatggggccaaaaggccattgaagtgcgctctttgcaatccagggttctggaaagtgagctgaagcgcaggtcaattaagaagctgagattcctgtgcacccggggtgacaag

SEQ ID NO: 96 - protein, 46 aa, Homo sapiens
FECTQRTTGWGQKAIEVRSLQSRVLESELKRRSIKKLRFLCTRGDK

SEQ ID NO: 97 - DNA, 93 bp, Homo sapiens
ctgttctttacctctaccctgcgcaatcaccacagccgggtttacttcatgacacttggaaaacttgaagagctccaaagcaattatgatgtc

SEQ ID NO: 98 - protein, 31 aa, Homo sapiens
LFFTSTLRNHHSRVYFMTLGKLEELQSNYDV

SEQ ID NO: 99 - DNA, 4500 bp, Homo sapiens
atggcgggacctgggggctggagggacagggaggtcacggatctgggccacctgccggatccaactggaatattctcactagataaaaccattggccttggtacttatggcagaatctatttgggacttcatgagaagactggtgcatttacagctgttaaagtgatgaacgctcgtaagacccctttacctgaaataggaaggcgagtgagagtgaataaatatcaaaaatctgttgggtggagatacagtgatgaggaagaggatctcaggactgaactcaaccttctgaggaagtactctttccacaaaaacattgtgtccttctatggagcatttttcaagctgagtccccctggtcagcggcaccaactttggatggtgatggagttatgtgcagcaggttcggtcactgatgtagtgagaatgaccagtaatcagagtttaaaagaagattggattgcttatatctgccgagaaatccttcagggcttagctcaccttcacgcacaccgagtaattcaccgggacatcaaaggtcagaatgtgctgctgactcataatgctgaagtaaaactggttgattttggagtgagtgcccaggtgagcagaactaatggaagaaggaatagtttcattgggacaccatactggatggcacctgaggtgattgactgtgatgaggacccaagacgctcctatgattacagaagtgatgtgtggtctgtgggaattactgccattgaaatggctgaaggagcccctcctctgtgtaaccttcaacccttggaagctctcttcgttattttgcgggaatctgctcccacagtcaaatccagcggatggtcccgtaagttccacaatttcatggaaaagtgtacgataaaaaatttcctgtttcgtcctacttctgcaaacatgcttcaacacccatttgttcgggatataaaaaatgaacgacatgttgttgagtcattaacaaggcatcttactggaatcattaaaaaaagacagaaaaaaggaatacctttgatctttgaaagagaagaagctattaaggaacagtacaccgtgagaagattcagaggaccctcttgcactcacgagcttctgagattgccaaccagcagcagatgcagaccacttagagtcctgcatggggaaccctctcagccaaggtggctacctgatcgagaagagccacaggtccaggcacttcagcagctacagggagcagccagggtattcatgccactgcaggctctggacagtgcacctaagcctctaaaggggcaggctcaggcacctcaacgactacaaggggcagctcgggtgttcatgccactacaggctcaggtgaaggctaaagcctctaaacctctacaaatgcagattaaggcacctccacgactacggagggcagccagggtgctcatgccactacaggcacaggttagggcacctaggcttctgcaggtacagtcccaggtatccaaaaagcagcaggcccagacccagacatcagaaccacaagatttggaccaggtaccagaggaatttcagggtcaagatcaggtacccgaacaacaaaggcagggccaggcccctgaacaacagcagaggcacaaccaggtgcctgaacaagagctggagcagaaccaggcacctgaacagccagaggtacaggaacaggctgccgagcctgcacaggcagagactgaggcagaggaacctgagtcattacgagtaaatgcccaggtatttctgcccctgctatcacaagatcaccatgtgctgttgccactacatttggatactcaggtgctcattccagtagaggggcaaactgaaggatcacctcaggcacaggcttggacactagaacccccacaggcaattggctcagttcaagcactgatagagggactatcaagagacttgcttcgggcaccaaactcaaataactcaaagccacttggtccgttgcaaaccctgatggaaaatctgtcatcaaataggttttactcacaaccagaacaggcacgggagaaaaaatcaaaagtttctactctgaggcaagcactggcaaaaagactatcaccaaagaggttcagggcaaagtcatcatggagacctgaaaagcttgaactctcggatttagaagcccgcaggcaaaggcgccaacgcagatgggaagatatctttaatcagcatgaggaagaattgagacaagttgataaagacaaagaagatgaatcatcagacaatgatgaagtatttcattcgattcaggctgaagtccagatagagccattgaagccatacatttcaaatcctaaaaaaattgaggttcaagagagatctccttctgtgcctaacaaccaggatcatgcacatcatgtcaagttctcttcaaggacatggcacatgcttttctgtcttttcattagcgttcctcagcggtctcttttggaacaagctcagaagcccattgacatcagacaaaggagttcgcaaaatcgtcaaaattggctggcagcatcagaatcttcttctgaggaagaaagtcctgtgactggaaggaggtctcagtcatcaccaccttattctactattgatcagaagttgctggttgacatccatgttccagatggatttaaagtaggaaaaatatcaccccctgtatacttgacaaacgaatgggtaggctataatgcactctctgaaatcttccggaatgattggttaactccggcacctgtcattcagccacctgaagaggatggtgattatgttgaactctatgatgccagtgctgatactgatggtgatgatgatgatgagtctaatgatacttttgaagatacctatgatcatgccaatggcaatgatgacttggataaccaggttgatcaggctaatgatgtttgtaaagaccatgatgatgacaacaataagtttgttgatgatgtaaataataattattatgaggcgcctagttgtccaagggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggaggtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatcttatacgctggattcgtagaagtacctgaggaatcacctaagcaaccctctgaagtcaatgttaacccactctatgtctctcctgcatgtaaaaaaccactaatccacatgtatgaaaaggagttcacttctgagatctgctgtggttctttgtggggagtcaatttgctgttgggaacccgatctaatctatatctgatggacagaagtggaaaggctgacattactaaacttataaggcgaagaccattccgccagattcaagtcttagagccactcaatttgctgattaccatctcaggtcataagaacagacttcgggtgtatcatctgacctggttgaggaacaagattttgaataatgatccagaaagtaaaagaaggcaagaagaaatgctgaagacagaggaagcctgcaaagctattgataagttaacaggctgtgaacacttcagtgtcctccaacatgaagaaacaacatatattgcaattgctttgaaatcatcaattcacctttatgcatgggcaccaaagtcctttgatgaaagcactgctattaaagtatttccaacacttgatcataagccagtgacagttgacctggctattggttctgaaaaaagactaaagattttcttcagctcagcagatggatatcacctcatcgatgcagaatctgaggttatgtctgatgtgaccctgccaaagaatcccctggaaatcattataccacagaatatcatcattttacctgattgcttgggaattggcatgatgctcaccttcaatgctgaagccctctctgtggaagcaaatgaacaactcttcaagaagatccttgaaatgtggaaagacataccatcttctatagcttttgaatgtacacagcgaaccacaggatggggccaaaaggccattgaagtgcgctctttgcaatccagggttctggaaagtgagctgaagcgcaggtcaattaagaagctgagattcctgtgcacccggggtgacaagctgttctttacctctaccctgcgcaatcaccacagccgggtttacttcatgacacttggaaaacttgaagagctccaaagcaattatgatgtc

SEQ ID NO: 100 - protein, 1500 aa, Homo sapiens
MAGPGGWRDREVTDLGHLPDPTGIFSLDKTIGLGTYGRIYLGLHEKTGAFTAVKVMNARKTPLPEIGRRVRVNKYQKSVGWRYSDEEEDLRTELNLLRKYSFHKNIVSFYGAFFKLSPPGQRHQLWMVMELCAAGSVTDVVRMTSNQSLKEDWIAYICREILQGLAHLHAHRVIHRDIKGQNVLLTHNAEVKLVDFGVSAQVSRTNGRRNSFIGTPYWMAPEVIDCDEDPRRSYDYRSDVWSVGITAIEMAEGAPPLCNLQPLEALFVILRESAPTVKSSGWSRKFHNFMEKCTIKNFLFRPTSANMLQHPFVRDIKNERHVVESLTRHLTGIIKKRQKKGIPLIFEREEAIKEQYTVRRFRGPSCTHELLRLPTSSRCRPLRVLHGEPSQPRWLPDREEPQVQALQQLQGAARVFMPLQALDSAPKPLKGQAQAPQRLQGAARVFMPLQAQVKAKASKPLQMQIKAPPRLRRAARVLMPLQAQVRAPRLLQVQSQVSKKQQAQTQTSEPQDLDQVPEEFQGQDQVPEQQRQGQAPEQQQRHNQVPEQELEQNQAPEQPEVQEQAAEPAQAETEAEEPESLRVNAQVFLPLLSQDHHVLLPLHLDTQVLIPVEGQTEGSPQAQAWTLEPPQAIGSVQALIEGLSRDLLRAPNSNNSKPLGPLQTLMENLSSNRFYSQPEQAREKKSKVSTLRQALAKRLSPKRFRAKSSWRPEKLELSDLEARRQRRQRRWEDIFNQHEEELRQVDKDKEDESSDNDEVFHSIQAEVQIEPLKPYISNPKKIEVQERSPSVPNNQDHAHHVKFSSRTWHMLFCLFISVPQRSLLEQAQKPIDIRQRSSQNRQNWLAASESSSEEESPVTGRRSQSSPPYSTIDQKLLVDIHVPDGFKVGKISPPVYLTNEWVGYNALSEIFRNDWLTPAPVIQPPEEDGDYVELYDASADTDGDDDDESNDTFEDTYDHANGNDDLDNQVDQANDVCKDHDDDNNKFVDDVNNNYYEAPSCPRASYGRDGSCKQDGYDGSRGKEEAYRGYGSHTANRSHGGSAASEDNAAIGDQEEHAANIGSERRGSEGDGGGGNEASNAIDSGAAPSAPDHESDNKDISESSTQSDFSANHSSPSKGSGMSADANFASAILYAGFVEVPEESPKQPSEVNVNPLYVSPACKKPLIHMYEKEFTSEICCGSLWGVNLLLGTRSNLYLMDRSGKADITKLIRRRPFRQIQVLEPLNLLITISGHKNRLRVYHLTWLRNKILNNDPESKRRQEEMLKTEEACKAIDKLTGCEHFSVLQHEETTYIAIALKSSIHLYAWAPKSFDESTAIKVFPTLDHKPVTVDLAIGSEKRLKIFFSSADGYHLIDAESEVMSDVTLPKNPLEIIIPQNIIILPDCLGIGMMLTFNAEALSVEANEQLFKKILEMWKDIPSSIAFECTQRTTGWGQKAIEVRSLQSRVLESELKRRSIKKLRFLCTRGDKLFFTSTLRNHHSRVYFMTLGKLEELQSNYDV

SEQ ID NO: 101 - DNA, 57 bp, Homo sapiens; identical to SEQ ID NO: 1

SEQ ID NO: 102 - protein, 19 aa, Homo sapiens
MAGPGGWRDREVTDLGHLP

SEQ ID NO: 103 - DNA, 66 bp, Homo sapiens; identical to SEQ ID NO: 3

SEQ ID NO: 104 - protein, 22 aa, Homo sapiens
DPTGIFSLDKTIGLGTYGRIYL

SEQ ID NO: 105 - DNA, 57 bp, Homo sapiens; identical to SEQ ID NO: 5

SEQ ID NO: 106 - protein, 19 aa, Homo sapiens
GLHEKTGAFTAVKVMNARK

SEQ ID NO: 107 - DNA, 72 bp, Homo sapiens; identical to SEQ ID NO: 7

SEQ ID NO: 108 - protein, 24 aa, Homo sapiens
TPLPEIGRRVRVNKYQKSVGWRYS

SEQ ID NO: 109 - DNA, 126 bp, Homo sapiens; identical to SEQ ID NO: 9

SEQ ID NO: 110 - protein, 42 aa, Homo sapiens; identical to SEQ ID NO: 10

SEQ ID NO: 111 - DNA, 111 bp, Homo sapiens; identical to SEQ ID NO: 11

SEQ ID NO: 112 - protein, 37 aa, Homo sapiens; identical to SEQ ID NO: 12

SEQ ID NO: 113 - DNA, 91 bp, Homo sapiens; identical to SEQ ID NO: 13

SEQ ID NO: 114 - protein, 31 aa, Homo sapiens; identical to SEQ ID NO: 14

SEQ ID NO: 115 - DNA, 131 bp, Homo sapiens; identical to SEQ ID NO: 15

SEQ ID NO: 116 - protein, 43 aa, Homo sapiens; identical to SEQ ID NO: 16

SEQ ID NO: 117 - DNA, 55 bp, Homo sapiens; identical to SEQ ID NO: 17

SEQ ID NO: 118 - protein, 19 aa, Homo sapiens
SDVWSVGITAIEMAEGAPP

SEQ ID NO: 119 - DNA, 79 bp, Homo sapiens; identical to SEQ ID NO: 19

SEQ ID NO: 120 - protein, 26 aa, Homo sapiens
LCNLQPLEALFVILRESAPTVKSSGW

SEQ ID NO: 121 - DNA, 176 bp, Homo sapiens; identical to SEQ ID NO: 21

SEQ ID NO: 122 - protein, 59 aa, Homo sapiens; identical to SEQ ID NO: 22

SEQ ID NO: 123 - DNA, 64 bp, Homo sapiens; identical to SEQ ID NO: 23

SEQ ID NO: 124 - protein, 21 aa, Homo sapiens
IPLIFEREEAIKEQYTVRRFR

SEQ ID NO: 125 - DNA, 1156 bp, Homo sapiens; identical to SEQ ID NO: 25

SEQ ID NO: 126 - protein, 385 aa, Homo sapiens; identical to SEQ ID NO: 26

SEQ ID NO: 127 - DNA, 108 bp, Homo sapiens; identical to SEQ ID NO: 27

SEQ ID NO: 128 - protein, 36 aa, Homo sapiens; identical to SEQ ID NO: 28

SEQ ID NO: 129 - DNA, 68 bp, Homo sapiens; identical to SEQ ID NO: 29

SEQ ID NO: 130 - protein, 23 aa, Homo sapiens
VQERSPSVPNNQDHAHHVKFSSS

SEQ ID NO: 131 - DNA, 260 bp, Homo sapiens
cgttcctcagcggtctcttttggaacaagctcagaagcccattgacatcagacaaaggagttcgcaaaatcgtcaaaattggctggcagcatcaggtgattcaaagcacaaaattttagcaggcaaaacacagagctactgtttaacaatttatatttcagaagtcaagaaagaagaatttcaagaaggaatgaatcaaaagtgtcagggagcccaagtaggattaggacctgaaggccattgtatttggcaattgggtg

SEQ ID NO: 132 - protein, 87 aa, Homo sapiens
VPQRSLLEQAQKPIDIRQRSSQNRQNWLAASGDSKHKILAGKTQSYCLTIYISEVKKEEFQEGMNQKCQGAQVGLGPEGHCIWQLGE

SEQ ID NO: 133 - DNA, 98 bp, Homo sapiens; identical to SEQ ID NO: 33

SEQ ID NO: 134 - protein, 32 aa, Homo sapiens; identical to SEQ ID NO: 34

SEQ ID NO: 135 - DNA, 365 bp, Homo sapiens; identical to SEQ ID NO: 35

SEQ ID NO: 136 - protein, 122 aa, Homo sapiens; identical to SEQ ID NO: 36

SEQ ID NO: 137 - DNA, 337 bp, Homo sapiens
ggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggaggtaagggagtcgttcgaaccagtgaagagagtggagcccttggactcaatggagaagaaaattgctcagagacagatggtccaggattgaagagacctgcgtctcaggactttgaatatctacaggag

SEQ ID NO: 138 - protein, 112 aa, Homo sapiens
ASYGRDGSCKQDGYDGSRGKEEAYRGYGSHTANRSHGGSAASEDNAAIGDQEEHAANIGSERRGSEGDGGKGVVRTSEESGALGLNGEENCSETDGPGLKRPASQDFEYLQE

SEQ ID NO: 139 - DNA, 187 bp, Homo sapiens
gagccaggtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatct

SEQ ID NO: 140 - protein, 63 aa, Homo sapiens
EPGGGNEASNAIDSGAAPSAPDHESDNKDISESSTQSDFSANHSSPSKGSGMSADANFASAIL

SEQ ID NO: 141 - DNA, 156 bp, Homo sapiens; identical to SEQ ID NO: 83

SEQ ID NO: 142 - protein, 52 aa, Homo sapiens; identical to SEQ ID NO: 84

SEQ ID NO: 143 - DNA, 144 bp, Homo sapiens; identical to SEQ ID NO: 85

SEQ ID NO: 144 - protein, 48 aa, Homo sapiens; identical to SEQ ID NO: 86

SEQ ID NO: 145 - DNA, 159 bp, Homo sapiens; identical to SEQ ID NO: 87

SEQ ID NO: 146 - protein, 53 aa, Homo sapiens; identical to SEQ ID NO: 88

SEQ ID NO: 147 - DNA, 101 bp, Homo sapiens; identical to SEQ ID NO: 89

SEQ ID NO: 148 - protein, 33 aa, Homo sapiens; identical to SEQ ID NO: 90

SEQ ID NO: 149 - DNA, 150 bp, Homo sapiens; identical to SEQ ID NO: 91

SEQ ID NO: 150 - protein, 50 aa, Homo sapiens; identical to SEQ ID NO: 92

SEQ ID NO: 151 - DNA, 160 bp, Homo sapiens; identical to SEQ ID NO: 93

SEQ ID NO: 152 - protein, 54 aa, Homo sapiens; identical to SEQ ID NO: 94

SEQ ID NO: 153 - DNA, 140 bp, Homo sapiens; identical to SEQ ID NO: 95

SEQ ID NO: 154 - protein, 46 aa, Homo sapiens; identical to SEQ ID NO: 96

SEQ ID NO: 155 - DNA, 93 bp, Homo sapiens; identical to SEQ ID NO: 97

SEQ ID NO: 156 - protein, 31 aa, Homo sapiens; identical to SEQ ID NO: 98

SEQ ID NO: 157 - DNA, 4767 bp, Homo sapiens
atggcgggacctgggggctggagggacagggaggtcacggatctgggccacctgccggatccaactggaatattctcactagataaaaccattggccttggtacttatggcagaatctatttgggacttcatgagaagactggtgcatttacagctgttaaagtgatgaacgctcgtaagacccctttacctgaaataggaaggcgagtgagagtgaataaatatcaaaaatctgttgggtggagatacagtgatgaggaagaggatctcaggactgaactcaaccttctgaggaagtactctttccacaaaaacattgtgtccttctatggagcatttttcaagctgagtccccctggtcagcggcaccaactttggatggtgatggagttatgtgcagcaggttcggtcactgatgtagtgagaatgaccagtaatcagagtttaaaagaagattggattgcttatatctgccgagaaatccttcagggcttagctcaccttcacgcacaccgagtaattcaccgggacatcaaaggtcagaatgtgctgctgactcataatgctgaagtaaaactggttgattttggagtgagtgcccaggtgagcagaactaatggaagaaggaatagtttcattgggacaccatactggatggcacctgaggtgattgactgtgatgaggacccaagacgctcctatgattacagaagtgatgtgtggtctgtgggaattactgccattgaaatggctgaaggagcccctcctctgtgtaaccttcaacccttggaagctctcttcgttattttgcgggaatctgctcccacagtcaaatccagcggatggtcccgtaagttccacaatttcatggaaaagtgtacgataaaaaatttcctgtttcgtcctacttctgcaaacatgcttcaacacccatttgttcgggatataaaaaatgaacgacatgttgttgagtcattaacaaggcatcttactggaatcattaaaaaaagacagaaaaaaggaatacctttgatctttgaaagagaagaagctattaaggaacagtacaccgtgagaagattcagaggaccctcttgcactcacgagcttctgagattgccaaccagcagcagatgcagaccacttagagtcctgcatggggaaccctctcagccaaggtggctacctgatcgagaagagccacaggtccaggcacttcagcagctacagggagcagccagggtattcatgccactgcaggctctggacagtgcacctaagcctctaaaggggcaggctcaggcacctcaacgactacaaggggcagctcgggtgttcatgccactacaggctcaggtgaaggctaaagcctctaaacctctacaaatgcagattaaggcacctccacgactacggagggcagccagggtgctcatgccactacaggcacaggttagggcacctaggcttctgcaggtacagtcccaggtatccaaaaagcagcaggcccagacccagacatcagaaccacaagatttggaccaggtaccagaggaatttcagggtcaagatcaggtacccgaacaacaaaggcagggccaggcccctgaacaacagcagaggcacaaccaggtgcctgaacaagagctggagcagaaccaggcacctgaacagccagaggtacaggaacaggctgccgagcctgcacaggcagagactgaggcagaggaacctgagtcattacgagtaaatgcccaggtatttctgcccctgctatcacaagatcaccatgtgctgttgccactacatttggatactcaggtgctcattccagtagaggggcaaactgaaggatcacctcaggcacaggcttggacactagaacccccacaggcaattggctcagttcaagcactgatagagggactatcaagagacttgcttcgggcaccaaactcaaataactcaaagccacttggtccgttgcaaaccctgatggaaaatctgtcatcaaataggttttactcacaaccagaacaggcacgggagaaaaaatcaaaagtttctactctgaggcaagcactggcaaaaagactatcaccaaagaggttcagggcaaagtcatcatggagacctgaaaagcttgaactctcggatttagaagcccgcaggcaaaggcgccaacgcagatgggaagatatctttaatcagcatgaggaagaattgagacaagttgataaagacaaagaagatgaatcatcagacaatgatgaagtatttcattcgattcaggctgaagtccagatagagccattgaagccatacatttcaaatcctaaaaaaattgaggttcaagagagatctccttctgtgcctaacaaccaggatcatgcacatcatgtcaagttctcttcaagcgttcctcagcggtctcttttggaacaagctcagaagcccattgacatcagacaaaggagttcgcaaaatcgtcaaaattggctggcagcatcaggtgattcaaagcacaaaattttagcaggcaaaacacagagctactgtttaacaatttatatttcagaagtcaagaaagaagaatttcaagaaggaatgaatcaaaagtgtcagggagcccaagtaggattaggacctgaaggccattgtatttggcaattgggtgaatcttcttctgaggaagaaagtcctgtgactggaaggaggtctcagtcatcaccaccttattctactattgatcagaagttgctggttgacatccatgttccagatggatttaaagtaggaaaaatatcaccccctgtatacttgacaaacgaatgggtaggctataatgcactctctgaaatcttccggaatgattggttaactccggcacctgtcattcagccacctgaagaggatggtgattatgttgaactctatgatgccagtgctgatactgatggtgatgatgatgatgagtctaatgatacttttgaagatacctatgatcatgccaatggcaatgatgacttggataaccaggttgatcaggctaatgatgtttgtaaagaccatgatgatgacaacaataagtttgttgatgatgtaaataataattattatgaggcgcctagttgtccaagggcaagctatggcagagatggaagctgcaagcaagatggttatgatggaagtcgtggaaaagaggaagcctacagaggctatggaagccatacagccaatagaagccatggaggaagtgcagccagtgaggacaatgcagccattggagatcaggaagaacatgcagccaatataggcagtgaaagaagaggcagtgagggtgatggaggtaagggagtcgttcgaaccagtgaagagagtggagcccttggactcaatggagaagaaaattgctcagagacagatggtccaggattgaagagacctgcgtctcaggactttgaatatctacaggaggagccaggtggtggaaatgaggcctcaaatgccattgactcaggtgctgcaccgtcagcacctgatcatgagagtgacaataaggacatatcagaatcatcaacacaatcagatttttctgccaatcactcatctccttccaaaggttctgggatgtctgctgatgctaactttgccagtgccatcttatacgctggattcgtagaagtacctgaggaatcacctaagcaaccctctgaagtcaatgttaacccactctatgtctctcctgcatgtaaaaaaccactaatccacatgtatgaaaaggagttcacttctgagatctgctgtggttctttgtggggagtcaatttgctgttgggaacccgatctaatctatatctgatggacagaagtggaaaggctgacattactaaacttataaggcgaagaccattccgccagattcaagtcttagagccactcaatttgctgattaccatctcaggtcataagaacagacttcgggtgtatcatctgacctggttgaggaacaagattttgaataatgatccagaaagtaaaagaaggcaagaagaaatgctgaagacagaggaagcctgcaaagctattgataagttaacaggctgtgaacacttcagtgtcctccaacatgaagaaacaacatatattgcaattgctttgaaatcatcaattcacctttatgcatgggcaccaaagtcctttgatgaaagcactgctattaaagtatttccaacacttgatcataagccagtgacagttgacctggctattggttctgaaaaaagactaaagattttcttcagctcagcagatggatatcacctcatcgatgcagaatctgaggttatgtctgatgtgaccctgccaaagaatcccctggaaatcattataccacagaatatcatcattttacctgattgcttgggaattggcatgatgctcaccttcaatgctgaagccctctctgtggaagcaaatgaacaactcttcaagaagatccttgaaatgtggaaagacataccatcttctatagcttttgaatgtacacagcgaaccacaggatggggccaaaaggccattgaagtgcgctctttgcaatccagggttctggaaagtgagctgaagcgcaggtcaattaagaagctgagattcctgtgcacccggggtgacaagctgttctttacctctaccctgcgcaatcaccacagccgggtttacttcatgacacttggaaaacttgaagagctccaaagcaattatgatgtc

SEQ ID NO: 158 - protein, 1589 aa, Homo sapiens
MAGPGGWRDREVTDLGHLPDPTGIFSLDKTIGLGTYGRIYLGLHEKTGAFTAVKVMNARKTPLPEIGRRVRVNKYQKSVGWRYSDEEEDLRTELNLLRKYSFHKNIVSFYGAFFKLSPPGQRHQLWMVMELCAAGSVTDVVRMTSNQSLKEDWIAYICREILQGLAHLHAHRVIHRDIKGQNVLLTHNAEVKLVDFGVSAQVSRTNGRRNSFIGTPYWMAPEVIDCDEDPRRSYDYRSDVWSVGITAIEMAEGAPPLCNLQPLEALFVILRESAPTVKSSGWSRKFHNFMEKCTIKNFLFRPTSANMLQHPFVRDIKNERHVVESLTRHLTGIIKKRQKKGIPLIFEREEAIKEQYTVRRFRGPSCTHELLRLPTSSRCRPLRVLHGEPSQPRWLPDREEPQVQALQQLQGAARVFMPLQALDSAPKPLKGQAQAPQRLQGAARVFMPLQAQVKAKASKPLQMQIKAPPRLRRAARVLMPLQAQVRAPRLLQVQSQVSKKQQAQTQTSEPQDLDQVPEEFQGQDQVPEQQRQGQAPEQQQRHNQVPEQELEQNQAPEQPEVQEQAAEPAQAETEAEEPESLRVNAQVFLPLLSQDHHVLLPLHLDTQVLIPVEGQTEGSPQAQAWTLEPPQAIGSVQALIEGLSRDLLRAPNSNNSKPLGPLQTLMENLSSNRFYSQPEQAREKKSKVSTLRQALAKRLSPKRFRAKSSWRPEKLELSDLEARRQRRQRRWEDIFNQHEEELRQVDKDKEDESSDNDEVFHSIQAEVQIEPLKPYISNPKKIEVQERSPSVPNNQDHAHHVKFSSSVPQRSLLEQAQKPIDIRQRSSQNRQNWLAASGDSKHKILAGKTQSYCLTIYISEVKKEEFQEGMNQKCQGAQVGLGPEGHCIWQLGESSSEEESPVTGRRSQSSPPYSTIDQKLLVDIHVPDGFKVGKISPPVYLTNEWVGYNALSEIFRNDWLTPAPVIQPPEEDGDYVELYDASADTDGDDDDESNDTFEDTYDHANGNDDLDNQVDQANDVCKDHDDDNNKFVDDVNNNYYEAPSCPRASYGRDGSCKQDGYDGSRGKEEAYRGYGSHTANRSHGGSAASEDNAAIGDQEEHAANIGSERRGSEGDGGKGVVRTSEESGALGLNGEENCSETDGPGLKRPASQDFEYLQEEPGGGNEASNAIDSGAAPSAPDHESDNKDISESSTQSDFSANHSSPSKGSGMSADANFASAILYAGFVEVPEESPKQPSEVNVNPLYVSPACKKPLIHMYEKEFTSEICCGSLWGVNLLLGTRSNLYLMDRSGKADITKLIRRRPFRQIQVLEPLNLLITISGHKNRLRVYHLTWLRNKILNNDPESKRRQEEMLKTEEACKAIDKLTGCEHFSVLQHEETTYIAIALKSSIHLYAWAPKSFDESTAIKVFPTLDHKPVTVDLAIGSEKRLKIFFSSADGYHLIDAESEVMSDVTLPKNPLEIIIPQNIIILPDCLGIGMMLTFNAEALSVEANEQLFKKILEMWKDIPSSIAFECTQRTTGWGQKAIEVRSLQSRVLESELKRRSIKKLRFLCTRGDKLFFTSTLRNHHSRVYFMTLGKLEELQSNYDV

SEQ ID NO: 159 - DNA, 20 bp, Artificial Sequence
  oligonucleotide primer
aggaacagtacaccgtgaga

SEQ ID NO: 160 - DNA, 20 bp, Artificial Sequence
  oligonucleotide primer
agtcgtggaggtgccttaat

SEQ ID NO: 161 - DNA, 20 bp, Artificial Sequence
  oligonucleotide primer
taggaaggcgagtgagagtg

SEQ ID NO: 162 - DNA, 20 bp, Artificial Sequence
  oligonucleotide primer
ccggtgaattactcggtgtg

SEQ ID NO: 163 - DNA, 19 bp, Artificial Sequence
  oligonucleotide primer
taatacgactcactatagg

SEQ ID NO: 164 - DNA, 18 bp, Artificial Sequence
  oligonucleotide primer
attaaccctcactaaagg